(19) United States
(12) Patent Application Publication
(10) Pub. No.: US 2011/0082205 A1
(43) Pub. Date: Apr. 7, 2011

(54) Docosahexaenoic Acid Gel Caps

(76) Inventors: Cynthia A. PANKER, Jessup, MD (US); Michael Ames Billard, Laurel, MD (US); Alan Ryan, Ellicott City, MD (US); Bindi Dangi, Elkridge, MD (US)

(21) Appl. No.: 12/896,763

(22) Filed: Oct. 1, 2010

Related U.S. Application Data
(60) Provisional application No. 61/247,944, filed on Oct. 1, 2009.

(51) Int. Cl. A61K 31/232; A61P 3/06

(52) U.S. Cl. 514/549

(57) ABSTRACT

The present invention is directed to an oral dosage form comprising a gel capsule comprising, (a) plasticizer; (b) gelatin; (c) water; and (d) greater than 850 mg DHA ethyl ester, wherein less than about 2% (wt/wt) of the total fatty acid content of the dosage form is EPA, wherein the total weight of the capsule is about 1 g and wherein the capsule is stable at about 25° C. at 60% relative humidity.

This application claims the benefit of the filing date of U.S. Appl. No. 61/247,944, filed Oct. 1, 2009.

### BACKGROUND OF THE INVENTION

1. Field of the Invention

The present invention is directed to an oral dosage form comprising a gel capsule comprising, (a) a plasticizer; (b) a gelatin; (c) water; and (d) greater than 850 mg docosahexaenoic (DHA) ethyl ester, wherein less than about 2% (wt/wt) of the total fatty acid content of the dosage form is eicosapentaenoic (EPA), wherein the total weight of the capsule is about 1 g.

2. Background Art

Triglycerides include a glycerol esterified to three fatty acids. In the human body, high levels of triglycerides in the bloodstream have been linked to atherosclerosis, and, by extension, the risk of heart disease and stroke, as well as diabetes mellitus, pancreatitis, chronic renal disease, and certain primary hyperlipidemias. High triglyceride levels have also been associated with obesity, depression, bipolar disorder, and other affective disorders. Glueck, C. J., et al., Am. J. Med. Sci. 308:218-225 (1994).

Clinical trials have demonstrated that omega-3 long-chain polyunsaturated fatty acids (LC-PUFA) lower triglyceride levels. Two particular polyunsaturated fatty acids that have been shown to have therapeutic efficacy of reducing triglyceride levels when used in combination include (all-Z) 5,8,11,14,17-eicosapentaenoic acid, hereinafter referred to as EPA, and (all-Z)-4,7,10,13,16,19-docosahexaenoic acid, hereinafter referred to as DHA. EPA is known to be a precursor in the biosynthesis of prostaglandin PGE₃. These LC-PUFA are commonly found together in fatty fish, such as tuna, salmon, and mackerel.

Early studies of LC-PUFAs focused primarily on the effects of EPA on triglyceride levels. The relative contribution of other LC-PUFAs remained to be defined. Additional data, however, demonstrate that DHA and EPA have important cardioprotective properties. See, e.g., Mori and Holub, J. Nutr 126:3032-3039 (1996).

Previously, it was difficult to obtain pure EPA and DHA since the main source of these fatty acids, which occur together, was from the fats and oils of fish and marine animals. Unfortunately, in these sources, other fatty acids were always present in larger amounts. Most methods for extracting EPA and DHA from other triglycerides have not been satisfactory for producing high purity fatty acids, thereby making clinical studies difficult to conduct.

### BRIEF SUMMARY OF THE INVENTION

The present invention is directed to an oral dosage form comprising a gel capsule comprising, (a) a plasticizer; (b) a gelatin; (c) water; and (d) greater than 850 mg DHA ethyl ester, wherein less than about 2% (wt/wt) of the total fatty acid content of the dosage form is EPA, wherein the total weight of the capsule is about 1 g.

In some embodiments, the plasticizer is glycerine or glycerol. In some embodiments, the capsule is stable at about 25° C. at 60% relative humidity. In some embodiments, the capsule is stable at 30° C. at 65% relative humidity. In some embodiments, the capsule is stable at 40° C. at 75% relative humidity.

In some embodiments the capsule disintegrate less than 30 minutes, particularly less than 15 minutes, particularly less that 11 minutes and particularly less than 9 minutes as measured by the method set forth in USP <701>.

The present invention is also directed to a method of reducing plasma triglyceride levels in a subject by administering DHA. The method can comprise administering daily to a subject a dosage form comprising docosahexaenoic acid (DHA) ester wherein less than about 2% (w/w/) of the total fatty acid content of the dosage form is eicosapentaenoic acid (EPA), wherein the DHA ester is derived from an algal source. In some embodiments, the method comprises administering daily to the subject a dosage form comprising docosahexaenoic acid ester wherein less than about 2% (w/w/) of the total fatty acid content of the dosage form is EPA, wherein the DHA ester is about 85% to about 99.5% (wt/wt) of the total fatty acid content of the dosage form, and wherein the DHA ester is derived from an algal source. In some embodiments, the method comprises administering daily to the subject a dosage form comprising about 200 mg to about 4 g of DHA ester wherein less than about 2% (w/w/) of the total fatty acid content of the dosage faun is EPA. In some embodiments, the foregoing methods also result in a lowering of the amount of total cholesterol in the subject.

In some embodiments of the invention, the DHA ester is a DHA alkyl ester, e.g., a DHA methyl ester, ethyl ester or propyl ester. The DHA ester can be derived from various sources. In some embodiments, the DHA ester is derived from an algal source, e.g., Crypthecodinium cohnii or Schizochytrium sp.

The DHA ester used in the methods of the present invention can be purified to various levels. In some embodiments, the DHA ester is about 85% to about 99.5% (wt/wt) of the total fatty acid content of the dosage form, or about 85% to about 95% (wt/wt) of the total fatty acid content of the dosage form. In some embodiments, the DHA ester comprises about 90%, about 95%, or about 98% of the total fatty acid content of the dosage form. In some embodiments, the dosage form comprises about 0.5 g to about 4 g of DHA ester, or about 1 g to about 2 g of DHA ester.

The methods of the present invention use a dosage form wherein less than about 2% (w/w/) of the total fatty acid content of the dosage form is EPA. In some embodiments, the EPA is less than 1.5% of the total fatty acid content of the dosage form. In some embodiments, the EPA is less than 1% of the total fatty acid content of the dosage form, less than 0.2% of the total fatty acid content of the dosage foam, or less than 0.01% of the total fatty acid content of the dosage form.

In some embodiments, additional fatty acids are present in the dosage form. For example, in some embodiments, the dosage form comprises 0.1% to 20% or about 0.1% to about 20% of one or more of the following fatty acids: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) docosapentaenoic acid 22:5n-3, 22:5w3 (DPAn3); (k) docosapentaenoic acid 22:5n-6, 22:5w6 (DPAn6); and (l) 4,7,10,13,16,19,22,25 octacosaoctaenoic acid (C28:8). In some embodiments, the dosage form comprises 1% to 5% of one or more of the following fatty acids: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) docosapentaenoic acid 22:5n-3, 22:5w3 (DPAn3); (k) docosapentaenoic acid 22:5n-6, 22:5w6 (DPAn6); and (l) 4,7,10,13,16,19,22,25 octacosaoctaenoic acid (C28:8). In some embodiments, the dosage form comprises less than 1% each of the following fatty acids: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) docosapentaenoic acid 22:5n-3, 22:5w3 (DPAn3); (k) docosapentaenoic acid 22:5n-6, 22:5w6 (DPAn6); and (l) 4,7,10,13,16,19,22,25 octacosaoctaenoic acid (C28:8). In some embodiments, the dosage form comprises 4,7,10,13,16,19,22,25 octacosaoctaenoic acid (C28:8) in an amount of from about 0.5% to about 3%, from about 1% to about 2%, or about 1.3% (wt/wt) of the total fatty acid content of the dosage form.

In some embodiments, the invention is directed to a method of reducing plasma triglyceride levels in a subject, wherein the subject has a chronic condition, e.g., chronic elevated triglyceride levels. Thus, the invention can be directed to administering daily to the subject a dosage form comprising DHA ester for the remainder of the subject's lifetime (i.e., chronic administration), from 1 year to 20 years, or from 1 year to 10 years. In some embodiments, the invention is directed to a method of reducing plasma triglyceride level in a subject, the method comprising administering daily to the subject a dosage form comprising about 200 mg to about 4 g of DHA ester wherein less than about 2% (w/w/) of the total fatty acid content of the dosage form is EPA. The dosage form can be administered daily for 4 to 28 consecutive days, or for 7 to 14 consecutive days. In certain aspects of the foregoing embodiments, the administration of DHA ester results in a reduction in the subject's total cholesterol levels. The triglyceride levels and/or cholesterol levels in a subject can be reduced relative to a subject that has not been administered a dosage form comprising DHA ester. For example, in some embodiments the triglyceride levels in a subject are reduced about 25% to about 75% by day 14, or about 30% to about 65% by day 14, relative to a subject that has not been administered a dosage form comprising DHA ester. In certain aspects of those embodiments in which the subject's total cholesterol is also lowered, the total cholesterol level in a subject is reduced by about 15%, about 20%, about 25%, about 40%, about 15% to about 25% by day 14, or about 20% to about 40% by day 28, relative to a subject that has not been administered a dosage form comprising DHA ester.

The methods of the present invention can include administration of the dosage form once daily. The dosage form may be a capsule or any other suitable solid form. In some embodiments, the dosage form is an oral dosage form, e.g., a tablet, pill, gel cap or caplet. In particular embodiments the oral dosage form is a capsule.

The present invention is also directed to an oral dosage form comprising: (a) about 1 g of DHA ester; wherein the DHA ester is about 85% to about 99.5% (wt/wt) of the total fatty acid content of the dosage form; (b) a pharmaceutically acceptable excipient; wherein the dosage form is substantially free of EPA, and wherein the DHA ester is derived from an algal source.

### BRIEF SUMMARY OF THE FIGURES

FIG. 1 represents the plasma DHA fatty acid area percent in rats fed either DHA-EE, DHASCO®, or Lovaza®.

FIG. 2 represents the mean DHA levels from plasma total lipids assays. The DHA-EE administered was prepared as in Example 1.

FIG. 3 represents the plasma EPA fatty acid area percent in rats fed either DHA-EE, DHASCO®, or Lovaza®.

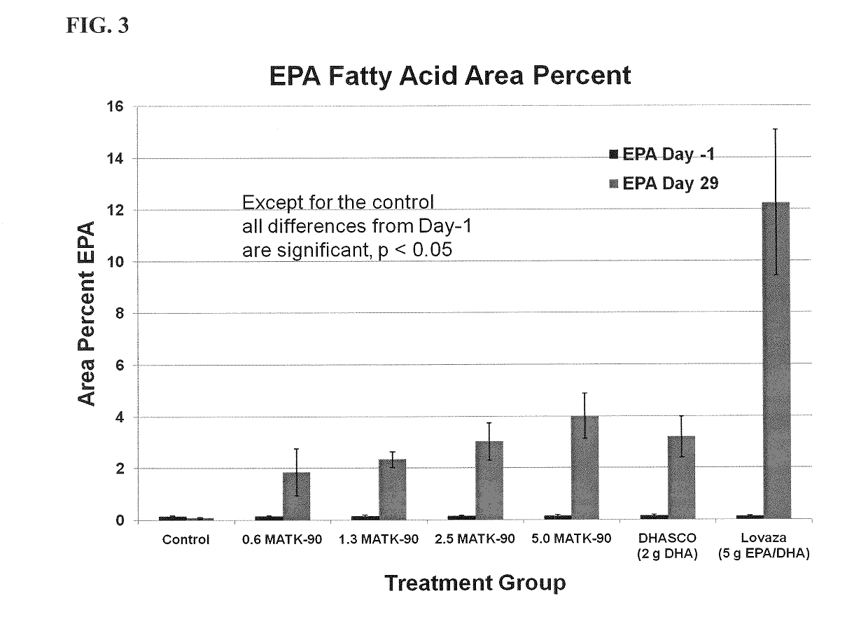

FIG. 4 represents the mean EPA levels from plasma total lipids assays. The DHA-EE administered was prepared as in Example 1.

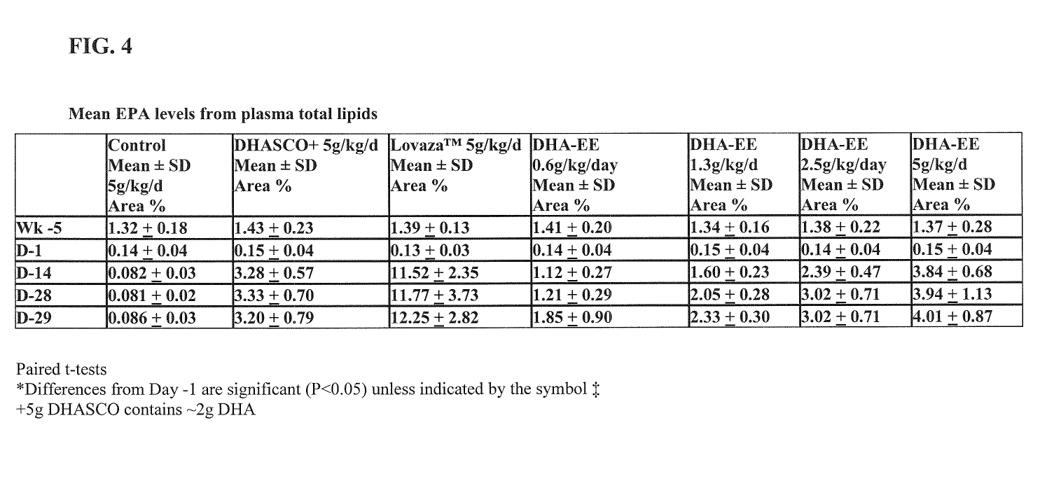

FIG. 5 represents a regression analysis of absolute change from baseline in triglyceride levels versus baseline triglyceride levels, wherein 1 to 6 g/day of DHA ethyl ester as prepared in Example 1 was used.

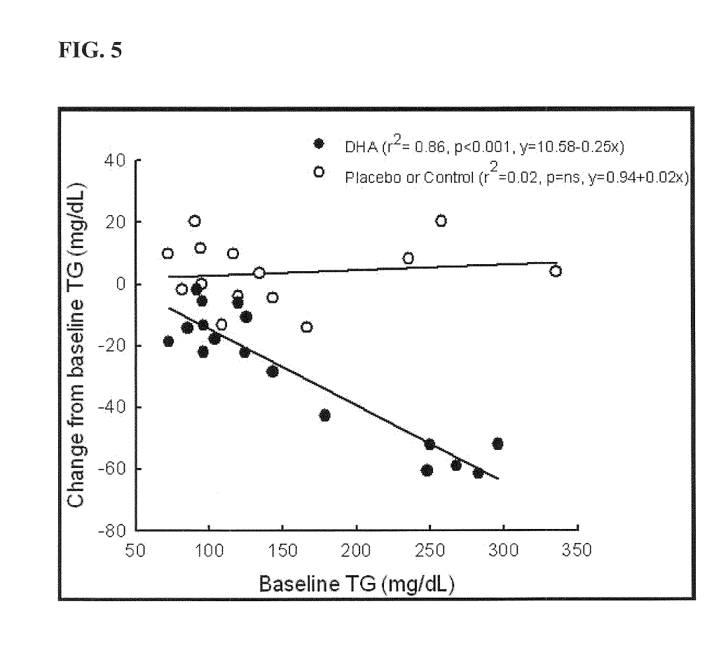

FIG. 6 depicts minicap batch hardness profiles for DHA-EE (09MC-20) and placebo (09MC-20P) obtained over the course of 19 drying days.

### DETAILED DESCRIPTION OF THE INVENTION

For the descriptions herein and the appended claims, the singular forms “a”, “an” and “the” include plural referents unless the context clearly indicates otherwise. Thus, for example, reference to “a compound” can refer to one or more than one compound.

Also, the use of “or” means “and/or” unless stated otherwise. Similarly, “comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

In reference to the present disclosure, the technical and scientific terms used in the descriptions herein will have the meanings commonly understood by one of ordinary skill in the art, unless specifically defined otherwise.

In the methods herein, a human subject suffering from the above disorders can be treated by administering a composition comprising DHA. In some embodiments, the present invention is directed to methods of reducing plasma triglyceride level in a subject using DHA ester. In some embodiments, the foregoing methods also result in a lowering of the amount of total cholesterol levels in the subject.

DHA is an ω-3 polyunsaturated fatty acid. As used herein, “DHA” refers to docosahexaenoic acid, also known by its chemical name (all-Z)-4,7,10,13,16,19-docosahexaenoic acid, as well as any salts or derivatives thereof. Thus, the term “DHA” encompasses the free acid DHA as well as DHA alkyl esters and triglycerides containing DHA. DHA is an ω-3 polyunsaturated fatty acid. Hence, in various embodiments, the DHA used in the method may be in the form of a phospholipid, a triglyceride, free fatty acid, or an alkyl ester. In some embodiments, the alkyl ester may comprise DHA methyl ester, ethyl ester, or propyl ester, as further described below.

Any source of DHA can be used in the compositions and methods described herein, including, for example, animal, plant and microbial sources. In some embodiments, a source of oils containing DHA suitable for the compositions and methods described herein is an animal source. Examples of animal sources include aquatic animals (e.g., fish, marine mammals; crustaceans such as krill and other euphausids; rotifers, etc.) and lipids extracted from animal tissues (e.g., brain, liver, eyes, etc.) and animal products such as eggs or milk. Examples of plant sources include macroalgae, flaxseeds, rapeseeds, corn, evening primrose, soy and borage. Examples of microorganisms include macroalgae, protists, bacteria and fungi (including yeast). For example, the DHA may be purified from fish oil, plant oil, seed oil, or other naturally occurring oils such that the DHA to EPA ratio are within the scope described herein.

In some embodiments, the composition of DHA is a microbial oil or is derived from microbial oil. Exemplary microbes from which microbial oil may be obtained, include, among others, the microbial groups Stramenopiles, Thraustochytrids, and Labrinthulids. Stramenopiles includes microalgae and algae-like microorganisms, including the following groups of microorganisms: Hamatores, Proteromonads, Opalines, Develpayella, Diplophrys, Labrinthulids, Thraustochytrids, Biosecids, Oomycetes, Hypochytridiomycetes, Commation, Reticulosphaera, Pelagomonas, Pelagococcus, Ollicola, Aureococcus, Parmales, Diatoms, Xanthophytes, Phaeophytes (brown algae), Eustigmatophytes, Raphidophytes, Synurids, Axodines (including Rhizochromulinaales, Pedinellales, Dictyochales), Chrysomeridales, Sarcinochrysidales, Hydrurales, Hibberdiales, and Chromulinales. The Thraustochytrids include the genera Schizochytrium (species include aggregatum, limnaceum, mangrovei, minutum, octosporum), Thraustochytrium (species include arudimentale, aureum, benthicola, globosum, kinnei, motivum, multirudimentale, pachydermum, proliferum, roseum, striatum), Ulkenia (species include amoeboidea, kerguelensis, minuta, profunda, radiate, sailens, sarkariana, schizochytrops, visurgensis, yorkensis), Aplanochytrium (species include haliotidis, kerguelensis, profunda, stocchinoi), Japonochytrium (species include marinum), Althornia (species include crouchii), and Elina (species include marisalba, sinorifica). The Labrinthulids include the genera Labyrinthula (species include algeriensis, coenocystis, chattonii, macrocystis, macrocystis atlantica, macrocystis macrocystis, marina, minuta, roscoffensis, valkanovii, vitellina, vitellina pacifica, vitellina vitellina, zopfi), Labyrinthomyxa (species include marina), Labyrinthuloides (species include haliotidis, yorkensis), Diplophrys (species include archeri), Pyrrhosorus* (species include marinus), Sorodiplophrys* (species include stercorea), and Chlamydomyxa* (species include labyrinthuloides, montana) (*=there is no current general consensus on the exact taxonomic placement of these genera).

In some embodiments, the microbial oil source is oleaginous microorganisms, such as certain marine algae. As used herein, “oleaginous microorganisms” are defined as microorganisms capable of accumulating greater than 20% of the dry weight of their cells in the form of lipids. In some embodiments, the DHA is derived from a phototrophic or heterotrophic single cell organism or multicellular organism, e.g., an algae. For example, the DHA may be derived from a diatom, e.g., a marine dinoflagellates (algae), such as Crypthecodinium sp., Thraustochytrium sp., Schizochytrium sp., or combinations thereof. Exemplary samples of C. cohnii, have been deposited with the American Type Culture Collection at Rockville, Md., and assigned the accession numbers 40750, 30021, 30334-30348, 3054130543, 30555-30557, 30571, 30572, 30772-30775, 30812, 40750, 50050-50060, and 50297-50300.

As used herein, the term microorganism, or any specific type of organism, includes wild strains, mutants or recombinant types. Organisms which can produce an enhanced level of oil containing DHA are considered to be within the scope of this invention. For example, cultivation of dinoflagellates such as C. cohnii has been described previously. See, e.g., U.S. Pat. No. 5,492,938 and Henderson et al., Phytochemistry 27:1679-1683 (1988). Also included are microorganisms designed to efficiently use more cost-effective substrates while producing the same amount of DHA as the comparable wild-type strains.

Organisms useful in the production of DHA can also include any manner of transgenic or other genetically modified organisms, such as a genetically modified plant or a genetically modified microorganism manipulated to produce DHA. e.g., plants, grown either in culture fermentation or in crop plants, e.g., cereals such as maize, barley, wheat, rice, sorghum, pearl millet, corn, rye and oats; or beans, soybeans, peppers, lettuce, peas, Brassica species (e.g., cabbage, broccoli, cauliflower, brussel sprouts, rapeseed, and radish), carrot, beets, eggplant, spinach, cucumber, squash, melons, cantaloupe, sunflowers, safflower, canola, flax, peanut, mustard, rapeseed, chickpea, lentil, white clover, olive, palm, borage, evening primrose, linseed, and tobacco. In some embodiments, the DHA is derived from a soybean source, including wild type and genetically modified soybean sources.

In some embodiments, the DHA may be purified in the form of free fatty acids, fatty acid esters, phospholipids, triglycerides, diglycerides, monoglycerides or combinations thereof by any means known to those of skill in the art. In some embodiments, the DHA comprises an ester. The term “ester” refers to the replacement of the hydrogen in the carboxylic acid group of the DHA molecule with another substituent. Typical esters are known to those in the art, a discussion of which is provided by Higuchi, T. and V. Stella in “Pro-drugs as Novel Delivery Systems,” Vol. 14, A.C.S. Symposium Series, Bioreversible Carriers in Drug Design, Ed. Edward B. Roche, American Pharmaceutical Association, Pergamon Press, 1987, and Protective Groups in Organic Chemistry, McOmie ed., Plenum Press, New York, 1973. In some embodiments, the ester is an alkyl ester. Examples of more common esters include C₁-C₆ esters, e.g., methyl, ethyl, propyl, butyl, pentyl, hexyl, or branched variations thereof, e.g., isopropyl, isobutyl, isopentyl, or t-butyl. In some embodiments, the ester is a carboxylic acid protective ester group, esters with aralkyl (e.g., benzyl, phenethyl), esters with lower alkenyl (e.g., allyl, 2-butenyl), esters with lower-alkoxy-lower-alkyl (e.g., methoxymethyl, 2-methoxyethyl, 2-ethoxyethyl), esters with lower-alkanoyloxy-lower-alkyl (e.g., acetoxymethyl, pivaloyloxymethyl, 1-pivaloyloxyethyl), esters with lower-alkoxycarbonyl-lower-alkyl (e.g., methoxycarbonylmethyl, isopropoxycarbonylmethyl), esters with carboxy-lower alkyl (e.g., carboxymethyl), esters with lower-alkoxycarbonyloxy-lower-alkyl (e.g., 1-(ethoxycarbonyloxy)ethyl, 1-(cyclohexyloxycarbonyloxy)ethyl), esters with carbamoyloxy-lower alkyl (e.g., carbamoyloxymethyl), and the like. In some embodiments, the added substituent is a cyclic hydrocarbon group, e.g., C₁-C₆ cycloalkyl, or C₁-C₆ aryl ester. Other esters include nitrobenzyl, methoxybenzyl, benzhydryl, and trichloroethyl. In some embodiments, the ester substituent is added to a DHA free acid molecule when the DHA is in a purified or semi-purified state. Alternatively, the DHA ester is formed upon conversion of a triglyceride to a ester. One of skill in the art can appreciate that some non-esterified DHA molecules can be present in the DHA compositions, e.g., DHA molecules that have not been esterified, or DHA triglyceride ester linkages that have been cleaved, e.g., hydrolyzed. In some embodiments, the non-esterified DHA molecules or the DHA triglyceride molecules constitute less than 3% (mol/mol), about 0.01% to about 2% (mol/mol), about 0.05% to about 1% (mol/mol), or about 0.01% to about 0.5% (mol/mol) of the total DHA molecules. In some embodiments, the amount of ethyl ester of DHA in the compositions may be at least about 91, 92, 93, 94, 95, 96, 97, 98, or 99% (wt/wt).

In some embodiments, the DHA of the present invention is a triglyceride, diglyceride or monoglyceride. A “triglyceride” is a glyceride in which the glycerol is esterified with three fatty acid groups. Typical triglycerides are known to those in the art. In some embodiments, the DHA is in the form of a triglyceride or a diglyceride, wherein one or more fatty acid groups other than DHA are present in the triglyceride or diglyceride. In some embodiments, DHA is the only fatty acid group on a triglyceride or diglyceride molecule. In some embodiments, one or more fatty acid groups of a triglyceride have been hydrolyzed, or cleaved.

In some embodiments, the DHA of the present invention is in the form of free fatty acid. “Free fatty acid” refers to fatty acid compounds in their acidic state, and salt derivatives thereof.

In the embodiments described herein, the composition of DHA for use in the methods may be obtained by standard techniques known in the art. In some embodiments, EPA may be removed during the purification of DHA, or alternatively, the DHA may be from an organism that produces DHA with the levels of EPA described herein, for example a production organism is selected that produces DHA with an insubstantial amount of EPA. DHA can be purified to various levels. DHA purification can be achieved by any means known to those of skill in the art, and can include the extraction of total oil from an organism which produces DHA. In some embodiments, EPA, ARA, and/or DPAn6 are then removed from the total oil, for example, via chromatographic methods. Alternatively, DHA purification can be achieved by extraction of total oil from an organism which produces DHA, but produces little, if any, amount of EPA, ARA, DPAn6, and/or flavonoids. In some embodiments, the oil can be diluted with other oils, such as sunflower oil to achieve the desired concentration of fatty acids.

Microbial oils useful in the present invention can be recovered from microbial sources by any suitable means known to those in the art. For example, the oils can be recovered by extraction with solvents such as chloroform, hexane, methylene chloride, methanol and the like, or by supercritical fluid extraction. Alternatively, the oils can be extracted using extraction techniques, such as are described in U.S. Pat. No. 6,750,048 and International Pub. No. WO 2001/053512, both filed Jan. 19, 2001, and entitled “Solventless extraction process,” both of which are incorporated herein by reference in their entirety. Processes for the preparation of various forms of DHA are also described in, among others, US Pub. No. 2009/0023808 “Production and Purification of Esters of Polyunsaturated Fatty Acids” by Raman et al., and US Pub. No. 2007/0032548 “Polyunsaturated fatty acids for treatment of dementia and pre-dementia-related conditions” by Ellis, incorporated herein by reference.

Additional extraction and/or purification techniques are taught in International Pub. No. WO 2001/076715; International Pub. No. WO 2001/076385; U.S. Pub. No. 2007/0004678; U.S. Pub. No. 2005/0129739; U.S. Pat. No. 6,399,803; and International Pub. No. WO 2001/051598; all of which are incorporated herein by reference in their entirety. The extracted oils can be evaporated under reduced pressure to produce a sample of concentrated oil material. Processes for the enzyme treatment of biomass for the recovery of lipids are disclosed in International Pub. No. WO 2003/092628; U.S. Pub. No. 2005/0170479; EP Pat. Pub. 0776356 and U.S. Pat. No. 5,928,696, the last two entitled “Process for extracting native products which are not water-soluble from native substance mixtures by centrifugal force,” all of which are incorporated herein by reference in their entirety.

In some embodiments, the DHA can be prepared as esters using a method comprising: a) reacting a composition comprising polyunsaturated fatty acids in the presence of an alcohol and a base to produce an ester of a polyunsaturated fatty acid from the triglycerides; and b) distilling the composition to recover a fraction comprising the ester of the polyunsaturated fatty acid, optionally wherein the method further comprises: c) combining the fraction comprising the ester of the polyunsaturated fatty acid with urea in a medium; d) cooling or concentrating the medium to form a urea-containing precipitate and a liquid fraction; and e) separating the precipitate from the liquid fraction. See, e.g., U.S. Pub. No. 2009/0023808, incorporated by reference herein in its entirety. In some embodiments, the purification process includes starting with refined, bleached, and deodorized oil (RBD oil), then performing low temperature fractionation using acetone to provide a concentrate. The concentrate can be obtained by base-catalyzed transesterification, distillation, and silica refining to produce the final DHA product.

Methods of determining purity levels of fatty acids are known in the art, and may include, e.g., chromatographic methods such as, e.g., HPLC silver ion chromatographic columns. Alternatively, purity levels may be determined by gas chromatography, with or without converting DHA to the corresponding alkyl ester. The percentage of fatty acids may also be determined using Fatty Acid Methyl Ester (FAME) analysis.

In some embodiments, the DHA esters can be derived from undiluted oil from a single cell microorganism, and in some embodiments, from undiluted DHASCO-T® (Martek Biosciences Corporation, Columbia, Md.). In some embodiments, the oil from which DHA compositions can be derived includes single cell microorganism oils that are manufactured by a controlled fermentation process followed by oil extraction and purification using methods common to the vegetable oil industry. In certain embodiments, the oil extraction and purification steps can include refining, bleaching, and deodorizing. In some embodiments, the undiluted DHA oil comprises about 40% to about 50% DHA by weight (about 400-500 mg DHA/g oil). In certain embodiments, the undiluted DHA oil can be enriched by cold fractionation (resulting in oil containing about 60% wt/wt of DHA triglyceride), which DHA fraction optionally can be transesterified, and subjected to further downstream processing to produce the active DHA of the invention. In some embodiments of the invention, downstream processing of the oil comprises distillation and/or silica refinement.

Thus, to produce oil from which DHA can be derived, in certain aspects, the following steps can be used: fermentation of a DHA producing microorganism; harvesting the biomass; spray drying the biomass; extracting oil from the biomass; refining the oil; bleaching the oil; chill filtering the oil; deodorizing the oil; and adding an antioxidant to the oil. In some embodiments, the microorganism culture can be progressively transferred from smaller scale fermenters to a production size fermenter. In some embodiments, following a controlled growth over a pre-established period, the culture can be harvested by centrifugation then pasteurized and spray dried. In certain embodiments, the dried biomass can be flushed with nitrogen and packaged before being stored frozen at −20° C. In certain embodiments, the DHA oil can be extracted from the dried biomass by mixing the biomass with n-hexane or isohexane in a batch process which disrupts the cells and allows the oil and cellular debris to be separated. In certain embodiments, the solvent can then be removed.

In some embodiments, the crude DHA oil can then undergo a refining process to remove free fatty acids and phospholipids. The refined DHA oil can be transferred to a vacuum bleaching vessel to assist in removing any remaining polar compounds and pro-oxidant metals, and to break down lipid oxidation products. The refined and bleached DHA oil can undergo a final clarification step by chilling and filtering the oil to facilitate the removal of any remaining insoluble fats, waxes, and solids.

Optionally, the DHA can be deodorized under vacuum in a packed column, counter current steam stripping deodorizer. Antioxidants such as ascorbyl palmitate, alpha-tocopherol, and tocotrienols can optionally be added to the deodorized oil to help stabilize the oil. In some embodiments, the final, undiluted DHA oil is maintained frozen at −20° C. until further processing.

In some embodiments, the DHA oil can be converted to DHA ester by methods known in the art. In some embodiments, DHA esters of the invention can be produced from DHA oil by the following steps: cold fractionation and filtration of the DHA oil (to yield for example about 60% triglyceride oil); direct transesterification (to yield about 60% DHA ethyl ester); molecular distillation (to yield about 88% DHA ethyl ester); silica refinement (to yield about 90% DHA ethyl ester); and addition of an antioxidant.

In some embodiments, the cold fractionation step can be carried out as follows: undiluted DHA oil (triglyceride) at about 500 mg/g DHA is mixed with acetone and cooled at a controlled rate in a tank with −80° C. chilling capabilities. Saturated triglycerides crystallize out of solution, while polyunsaturated triglycerides at about 600 mg/g DHA remain in the liquid state. The solids containing about 300 mg/g can be filtered out with a 20 micron stainless steel screen from the liquid stream containing about 600 mg/g DHA. The solids stream can then be heated (melted) and collected. The 600 mg/g DHA liquid stream can be desolventized with heat and vacuum and then transferred to the transesterification reactor.

In some embodiments, the transesterification step is carried out on the 600 mg/g DHA oil, wherein the transesterification is done via direct transesterification using ethanol and sodium ethoxide. The transesterified material (DHA-ethyl ester) can then be subject to molecular distillation and thus, further distilled (3 passes, heavies, lights, heavies) to remove most of the other saturated fatty acids and some sterols and non-saponifiable material. The DHA-ethyl ester (DHA-EE) can be further refined by passing it through a silica column.

DHA free fatty acids (DHA-FFA) can be made using, for example, the DHA containing oils described above. In some embodiments, the DHA-FFA can be obtained from DHA esters. DHA triglycerides, for example, can be saponified followed by a urea adduction step to make free fatty acids.

In some embodiments of the method, the DHA composition used has a level of DHA that is at least 85% (wt/wt) of total wt of fatty acid content; at least 90% (wt/wt) of total wt of fatty acid content; at least 95% (wt/wt) of total wt of fatty acid content; at least 96% (wt/wt) of total wt of fatty acid content; at least 97% (wt/wt) of total wt of fatty acid content; at least 98% (wt/wt) of total wt of fatty acid content; or at least 99% (wt/wt) of total wt of fatty acid content.

In some embodiments, DHA is present in an amount of about 85% to about 95% (wt/wt) of the total fatty acid content of the dosage form or unit dose. In some embodiments, the DHA is present in an amount greater about 85% (wt/wt) of the total fatty acid content of the dosage form or unit dose, greater than about 90% (wt/wt) of the total fatty acid content of the dosage form or unit dose, or greater than about 95% (wt/wt) of the total fatty acid content of the dosage form or unit dose. In some embodiments, the oil can be diluted with other oils, such as sunflower oil, to achieve the desired concentration of fatty acids.

In some embodiments, the DHA comprises about 85% to about 99.5% (wt/wt) of the total fatty acid content of the dosage form or unit dose.

In some embodiments, the DHA is greater than about 85% (wt/wt) of the total fatty acid content of the dosage form or unit dose, about 85% to about 99% (wt/wt) of the total fatty acid content of the dosage form or unit dose, about 87% to about 98% (wt/wt) of the total fatty acid content of the dosage form or unit dose, or about 90% to about 97% (wt/wt) of the total fatty acid content of the dosage form or unit dose. In some embodiments, the DHA is great than about 95%, about 97%, about 98%, about 99% or about 99.5% (wt/wt) of the total fatty acid content of the dosage form or unit dose.

In some embodiments, the DHA in the dosage form or unit dose comprises about 85% to about 96% of the total weight of the dosage form or unit dose.

In some of these embodiments, the DHA comprises about 85% to about 99.5% (wt/wt) of the total oil content of the dosage form or unit dose.

In some embodiments, the DHA is greater than about 85% (wt/wt) of the total oil content of the dosage form or unit dose, about 85% to 99.9% (wt/wt) of the total oil content of the dosage form or unit dose, about 85% to about 99% (wt/wt) of the total oil content of the dosage form or unit dose, about 87% to about 98% (wt/wt) of the total oil content of the dosage form or unit dose, or about 90% to about 97% (wt/wt) of the total oil content of the dosage form or unit dose. In some embodiments, the DHA is greater than about 95%, about 97%, about 98%, about 99% or about 99.5% (wt/wt) of the total oil content of the dosage form or unit dose. With respect to comparison of DHA to total fatty acid content or total oil content, weight % can be determined by calculating the area under the curve (AUC) using standard means, e.g., dividing the DHA AUC by the total fatty acid AUC.

As used herein, “or less” or “less than about” refers to percentages that include 0%, or amounts not detectable by current means. As used herein, “max” refers to percentages that include 0%, or amounts not detectable by current means.

The term “EPA” refers to eicosapentaenoic acid, known by its chemical name (all Z) 5,8,11,14,17-eicosapentaenoic acid, as well as any salts or derivatives thereof. Thus, the term “EPA” encompasses the free acid EPA as well as EPA alkyl esters and triglycerides containing EPA. EPA is an ω-3 polyunsaturated fatty acid. In some embodiments, the composition of DHA is substantially free of EPA. In some embodiments, EPA is less than 2% of the total fatty acid content of the composition, less than 1% of the total fatty acid content of the composition, less than 0.5% of the total fatty acid content of the composition, less than 0.2% of the total fatty acid content of the composition, less than 0.1% or less than 0.01% of the total fatty acid content of the composition. In some embodiments, the EPA is not detectable in the composition using techniques known in the art. In some embodiments, the DHA composition has no EPA.

DHA can also be administered substantially free of arachidonic acid (ARA). ARA refers to the compound (all-Z)-5,8,11,14-eicosatetraenoic acid (also referred to as (5Z,8Z,11Z,14Z)-icosa-5,8,11,14-tetraenoic acid), as well as any salts or derivatives thereof. Thus, the term “ARA” encompasses the free acid ARA as well as ARA alkyl esters and triglycerides containing ARA. ARA is an ω-6 polyunsaturated fatty acid. DHA is “substantially free of ARA” when ARA is less than about 3% (wt/wt) of the total fatty acid content of the dosage form. In some embodiments, ARA comprises less than about 2% (wt/wt) of the total fatty acid content of the dosage form, less than 1% (wt/wt) of the total fatty acid content of the dosage form, less than 0.5% (wt/wt) of the total fatty acid content of the dosage form, less than 0.2% (wt/wt) of the total fatty acid content of the dosage form, less than 0.1% or less than 0.01% (wt/wt) of the total fatty acid content of the dosage form. In some embodiments, the dosage form has no detectable amount of ARA.

DHA can also be administered substantially free of docosapentaenoic acid 22:5 n-6 (DPAn6). The term “DPAn6” refers to docosapentaenoic acid, omega 6, known by its chemical name (all-Z)-4,7,10,13,16-docosapentaenoic acid, as well as any salts or esters thereof. The term “DPAn6” encompasses the free acid DPAn6 as well as DPAn6 alkyl esters and triglycerides containing DPAn6. DPAn6 is an ω-6 polyunsaturated fatty acid. DHA is “substantially free of DPAn6” when DPAn6 is less than about 3% (wt/wt) of the total fatty acid content of the dosage form. In some embodiments, DPAn6 comprises less than about 2% (wt/wt) of the total fatty acid content of the dosage form, less than 1% (wt/wt) of the total fatty acid content of the dosage form, less than 0.5% (wt/wt) of the total fatty acid content of the dosage form, less than 0.2% (wt/wt) of the total fatty acid content of the dosage form, or less than 0.01% (wt/wt) of the total fatty acid content of the dosage form. In some embodiments, the dosage form has no detectable amount of DPAn6.

In some embodiments, the dosage form of the present invention does not contain a measurable amount of docosapentaenoic acid 22:5n-3 (DPAn3); docosapentaenoic acid 22:5n-6 (DPAn6); and/or 4,7,10,13,16,19,22,25 octacosaoctaenoic acid (C28:8).

In some embodiments, the DHA is administered in the substantial absence of an additional therapeutic agent.

In some embodiments, the composition of DHA may include an additional lipid. As used herein, the term “lipid” includes phospholipids (PL); free fatty acids; esters of fatty acids; triacylglycerols (TAG); diacylglycerides; monoacylglycerides; phosphatides; waxes (esters of alcohols and fatty acids); sterols and sterol esters; carotenoids; xanthophylls (e.g., oxycarotenoids); hydrocarbons; and other lipids known to one of ordinary skill in the art. The lipid can be chosen to have minimal adverse health effects or minimally affect the effectiveness of DHA when administered in combination with DHA.

In some embodiments, the composition of DHA may include an additional unsaturated lipid. In some embodiments, the unsaturated lipid is a polyunsaturated lipid, such as an omega-3 fatty acid or omega-6 fatty acid. An exemplary omega-6 fatty acid that may be used in the composition is docosapentaenoic acid (DPA), including DPAn6 or DPAn3.

In the methods and compositions herein, additional fatty acids can be present in the dosage form or unit dose or composition. These fatty acids can include fatty acids that were not removed during the purification process, i.e., fatty acids that were co-isolated with DHA from an organism. In some embodiments, one or more non-DHA fatty acids can be added to the dosage form or unit dose to achieve a desired concentration of specific non-DHA fatty acids. Any of these fatty acids can be present in various concentrations. For example, in some embodiments, the dosage form or unit dose comprises 0.01% to about 4% (wt/wt) of oleic acid. In some embodiments, the dosage form or unit dose comprises 0.01% to 0.5% (wt/wt) of one or more of the following fatty acids: (a) capric acid; (b) lauric acid; (c) myristic acid; (d) palmitic acid; (e) palmitoleic acid; (f) heptadecanoic acid; (g) stearic acid; (h) oleic acid; (i) linoleic acid; (j) α-linolenic acid; (k) arachidic acid; (1) eicosenoic acid; (m) arachidonic acid; (n) erucic acid; (o) docosapentaenoic acid 22:5n-3 (DPAn3); and (p) nervonic acid. In some embodiments, a dosage form or unit dose comprises 0.01% to 0.1% (wt/wt) of one or more of the following fatty acids: (a) lauric acid; (b) heptadecanoic acid; (c) stearic acid; (d) arachidic acid; (e) eicosenoic acid; and (f) arachidonic acid. In some embodiments, a dosage form or unit dose comprises less than 0.5% (wt/wt) each of the following fatty acids: (a) capric acid; (b) lauric acid; (c) myristic acid; (d) palmitic acid; (e) palmitoleic acid; (0 heptadecanoic acid; (g) stearic acid; (h) linoleic acid; (i) α-linolenic acid; (j) arachidic acid; (k) eicosenoic acid; (l) arachidonic acid; (m) erucic acid; (n) docosapentaenoic acid 22:5n-3 (DPAn3); and (o) nervonic acid. In some embodiments, the dosage form or unit doses of the present invention do not contain a measurable amount of one or more of the following fatty acids: (a) capric acid; (b) linoleic acid; (c) α-linolenic acid; and (d) docosapentaenoic acid 22:5n-3 (DPAn3).

In some embodiments, the dosage form or unit dose comprises 0.1% to 60% (wt/wt) of one or more of the following fatty acids, or esters thereof: (a) capric acid; (b) lauric acid; (c) myristic acid; (d) palmitic acid, (e) palmitoleic acid; (f) stearic acid; (g) oleic acid; (h) linoleic acid; (i) α-linolenic acid; (j) docosapentaenoic acid 22:5n-3 (DPAn3); (k) docosapentaenoic acid 22:5n-6 (DPAn6); and (k) 4,7,10,13,16,19,22,25 octacosaoctaenoic acid (C28:8). In some embodiments, the dosage form or unit dose comprises 20% to 40% (wt/wt) of one or more of the following fatty acids, or esters thereof: (a) capric acid; (b) lauric acid; (c) myristic acid; (d) palmitic acid; (e) palmitoleic acid; (f) stearic acid; (g) oleic acid; (h) linoleic acid; (i) α-linolenic acid; j) docosapentaenoic acid 22:5n-3 (DPAn3); (k) docosapentaenoic acid 22:5n-6 (DPAn6); and (l) 4,7,10,13,16,19,22,25 octacosaoctaenoic acid (C28:8). In some embodiments, the dosage form or unit dose comprises less than 1% (wt/wt) each of the following fatty acids, or esters thereof: (a) capric acid; (b) lauric acid; (c) myristic acid; (d) palmitic acid, (e) palmitoleic acid; (f) stearic acid; (g) oleic acid; (h) linoleic acid; (i) α-linolenic acid; (j) docosapentaenoic acid 22:5n-3 (DPAn3); (k) docosapentaenoic acid 22:5n-6 (DPAn6); and (l) 4,7,10,13,16,19,22,25 octacosaoctaenoic acid (C28:8).

In some embodiments the dosage form comprises 0.1% to 20% of one or more of the following fatty acids: (a) capric acid; (b) lauric acid; (c) myristic acid; (d) palmitic acid; (e) palmitoleic acid; (f) stearic acid; (g) oleic acid; (h) linoleic acid; (i) α-linolenic acid; G) DPAn3 (22:5, n-3); (k) DPAn6 (22:5, n-6); and (l) 4,7,10,13,16,19,22,25 octacosaoctaenoic acid (C28:8). In some embodiments, the dosage form comprises 1% to 5% of one or more of the following fatty acids: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) DPAn3 (22:5, n-3); (k) DPAn6 (22:5, n-6); and (l) 4,7,10,13,16,19,22,25 octacosaoctaenoic acid (C28:8). In some embodiments, the dosage form comprises less than 1% each of the following fatty acids: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) docosapentaenoic acid 22:5n-3, 22:5w3 (DPAn3); (k) docosapentaenoic acid 22:5n-6, 22:5w6 (DPAn6); and (l) 4,7,10,13,16,19,22,25 octacosaoctaenoic acid (C28:8).

In some of embodiments of DHA dosage form described herein, the dosage form is characterized by one or more the following fatty acids (or esters thereof). The embodiments provided herein may further comprise about 2% or less (wt/wt) of capric acid (C10:0). The embodiments herein may further comprise about 6% or less (wt/wt) of lauric acid (C12:0). The embodiments herein may further comprise about 20% or less (wt/wt), or about 5% to about 20% (wt/wt) of myristic acid (C14:0). The embodiments herein may further comprise about 20% (wt/wt) or less, or about 5% to about 20% (wt/wt) of palmitic acid (C16:0). The embodiments herein may further comprise about 3% (wt/wt) or less of palmitoleic acid (C16:1n-7). The embodiments herein may further comprise about 2% (wt/wt) or less of stearic acid (C18:0). The embodiments herein may further comprise about 40% (wt/wt) or less, or about 10% to about 40% (wt/wt) of oleic acid (C18:1n-9). The embodiments herein may further comprise about 5% (wt/wt) or less of linoleic acid (C18:2). The embodiments herein may further comprise about 2% (wt/wt) or less of nervonic acid (C24:1). The embodiments herein may further comprise about 3% (wt/wt) or less of other fatty acids or esters thereof. The DHA dosage form with the preceding characteristics may be derived from DHASCO®, an oil derived from Crypthecodinium cohnii containing docosahexaenoic acid (DHA).

In some embodiments, an oil is characterized by one or more of the following fatty acids (or esters thereof), expressed as % (wt/wt) of the total fatty acid content. The embodiments provided herein may further comprise about 2% or less (wt/wt) of capric acid (C10:0). The embodiments provided herein may further comprise about 6% or less (wt/wt) of lauric acid (C12:0). The embodiments provided herein may further comprise about 20% or less, or about 10 to about 20% (wt/wt) of myristic acid (C14:0). The embodiments provided herein may further comprise about 15% or less, or about 5 to about 15% (wt/wt) of palmitic acid (C16:0). The embodiments provided herein may further comprise about 5% or less (wt/wt) of palmitoleic acid (C16:1n-7). The embodiments provided herein may further comprise about 2% or less (wt/wt) of stearic acid (C18:0). The embodiments provided herein may further comprise about 20% or less, or about 5% to about 20% (wt/wt) of oleic acid (C18:1n-9). The embodiments provided herein may further comprise about 2% or less (wt/wt) of linoleic acid (C18:2). The embodiments provided herein may further comprise about 2% or less (wt/wt) of nervonic acid (C24:1). The embodiments provided herein may further comprise about 3% or less (wt/wt) of other fatty acids. An oil with the preceding characteristics may be an oil derived from Crypthecodinium cohnii containing docosahexaenoic acid (DHA).

In some embodiments, an oil is characterized by one or more the following fatty acids (or esters thereof), expressed as % (wt/wt) of the total fatty acid content. The embodiments provided herein may further comprise about 0.1% or less (wt/wt) of myristic acid (C14:0) or is not detectable. The embodiments provided herein may further comprise about 0.5% or less (wt/wt) of palmitic acid (C16:0). The embodiments provided herein may further comprise about 0.5% or less (wt/wt) of palmitoleic acid (C16:1n-7). The embodiments provided herein may further comprise about 0.5% or less (wt/wt) of stearic acid (C18:0), or is not detectable. The embodiments provided herein may further comprise about 4% or less (wt/wt) of oleic acid (C18:1n-9). The embodiments provided herein may further comprise less than 0.1% (wt/wt) of linoleic acid (C18:2) or is not detectable. The embodiments provided herein may further comprise less than 0.1% (wt/wt) of eicosapentaenoic acid (C20:5) or is not detectable. The embodiments provided herein may further comprise about 2% or less (wt/wt) of decosapentaenoic acid (22:5n-3). The embodiments provided herein may further comprise about 1% or less (wt/wt) of octacosaoctaenoic acid (28:8 n-3). The embodiments provided herein may further comprise about 0.5% or less (wt/wt) of tetracosaenoic acid (24:1n9). The embodiments provided herein may further comprise about 1% or less (wt/wt) of other fatty acids. The DHA in oil with the preceding characteristics may be in the form of a DHA ester, preferably an alkyl ester, such as a methyl ester, ethyl ester, propyl ester, or combinations thereof, prepared from an algal oil prepared from the Crypthecodinium, cohnii sp.

In some embodiments, the dosage form comprises, measured in percentage of free fatty acid, about 35-65%, 40-55%, 35-57%, or 57-65% DHA (22:6 n-3); about 0-2% capric acid (10:0); about 0-6% lauric acid (12:0); about 10-20% myristic acid (14:0); about 5-15% palmitic acid (16:0); about 0-5% palmitoleic acid (16:1); about 0-2% stearic acid (18:0); about 5-20% or 5-25% oleic acid (18:1); about 0-2% linoleic acid (18:2); and about 0-2% nervonic acid (24:1, n-9). In one embodiment, such an oil is derived from a microorganism of the genus Thraustochytrium. In another embodiment, the free fatty acid content is less than 0.4%.

The present invention also provides compositions comprising at least about 40% (wt/wt) DHA and at least about 0.1% (wt/wt) of DPAn3). In some embodiments, the compositions comprise at least about 55%, about 56%, about 57%, about 58%, about 59%, about 60%, about 61%, about 62%, about 63%, about 64%, about 65% (wt/wt) DHA, optionally in triglyceride form, as a percentage of total fatty acids.

In some embodiments, the DHA composition may comprise an oil derived from DHASCO®. DHASCO® is an oil derived from Crypthecodinium cohnii containing high amounts of docosahexaenoic acid (DHA), and more specifically contains the following approximate exemplary amounts of these fatty acids, as a percentage of the total fatty acids: myristic acid (14:0) 10-20%; palmitic acid (16:0) 10-20%; palmitoleic acid (16:1) 0-2%; stearic acid (18:0) 0-2%; oleic acid (18:1) 10-30%; linoleic acid (18:2) 0-5%; arachidic acid (20:0) 0-1%; behenic acid (22:0) 0-1%; docosapentaenoic acid (22:5) 0-1%; docosahexanoic acid (22:6) (DHA) 40-45%; nervonic acid (24:1) 0-2%; and others 0-3%.

In other embodiments, the compositions comprise at least about 90, 91, 92, 93, 94, 95, 96, 97, 98, or 99% (wt/wt) of DHA, optionally in ethyl ester form, as a percentage of total fatty acids. In certain embodiments, the amount of C28:8 in the compositions may be at least about 0.2, 0.3, 0.4, 0.5, 0.6, 0.7, 0.8, 0.9, 1.0, 1.1, 1.2, 1.3, 1.4 or 1.5% (wt/wt). The C28:8 may be present in any form, including triglyceride or ester form. For example, the C28:8 may be present in ethyl ester form.

In other embodiments, the compositions comprise at least about 90, 91, 92, 93, 94, 95, 96, 97, 98, or 99% (wt/wt) of DHA, optionally in ethyl ester form, as a percentage of total fatty acids. In certain embodiments, the amount of DPAn3 in the compositions may be at least about 0.1, 0.2, 0.3, 0.4, 0.5, 0.6, 0.7, 0.8, 0.9, or 1.0% (wt/wt) of DPAn3. The DPAn3 may be present in triglyceride or ester form. For example, the DPAn3 may be present in ethyl ester form. In certain embodiments, the compositions comprise all three of the DHA, C28:8 and DPAn3 in the concentration ranges specified above.

In further embodiments, the compositions may comprise less than about 1.0, 0.9, 0.8. 0.7, 0.6, 0.5, 0.4, 0.3, 0.2, or 0.1% (wt/wt) EPA in addition to the DHA and C28:8. In one embodiment, the compositions may comprise less than about 0.25% (wt/wt) EPA. The EPA may be present in any form, including triglyceride or ester form. In some embodiments, the compositions may comprise 0% (wt/wt) EPA.

Compositions useful in the methods herein also include compositions that comprise at least about 90% (wt/wt) of a combination of DPAn6 and DHA. In certain embodiments, the compositions may comprise at least about 91, 92, 93, 94, 95, 96, 97, 98, or 99% (wt/wt) of a combination of DPAn6 and DHA.

The present invention also provides compositions comprising at least about 90% (wt/wt) of a combination of DPAn6 and DHA, and at least one additional fatty acid or a derivative, such as an ester, thereof. In certain embodiments, the compositions may comprise at least about 91, 92, 93, 94, 95, 96, 97, 98, or 99% (wt/wt) of a combination of DPAn6 and DHA. In some embodiments, the additional fatty acid may have a boiling point of about 150-170° C. at a pressure of 0.8 mm Hg.

The DHA/DPAn6 compositions described above may further comprise less than about 4% of a saturated fatty acid or an ester thereof. In certain embodiments, the compositions may comprise less than about 3.5%, 3.0%, 2.5%, 2.0%, 1.5%, 1.0% or 0.5% of a saturated fatty acid or a derivative thereof.

The present invention also provides compositions comprising at least about 90% (wt/wt) of DHA and at least one additional fatty acid or a derivative thereof. In some embodiments, the amount of DHA in the compositions may be at least about 91, 92, 93, 94, 95, 96, 97, 98, or 99% (wt/wt). In certain embodiments, the additional fatty acid may have a boiling point of about 150-170° C. at a pressure of 0.8 mm Hg.

An exemplary DHA-containing oil derived from the algal oil of Crypthecodinium Cohnii, wherein the DHA comprises an ethyl ester, can be characterized by the specified amount of components listed in Table 1, where “Max” refers to the amount of the component that can be present up to the specified amount.

TABLE 1

| DHA content (mg/g) | 855-945 | |
|---|---|---|
| Fatty Acid Content: % of total EE | | |
| Eicosapentaenoic Acid (20:5ω3) | ND | |
| Myristic Acid (14:0) | 0.1% | |
| Palmitic Acid (16:0) | 0.5% | |
| Palmitoleic Acid (16:1ω7) | 0.4% | |
| Stearic Acid (18:0) | ND | |
| Oleic Acid (18:1ω9) | 4% | |
| Linoleic Acid (18:2ω6) | ND | |
| Docosapentaenoic acid (22:5ω3) | 1.3% | |
| Octacosaoctaenoic acid (28:8ω3) | 0.9% | |
| Tetracosaenoic Acid (24:1ω9) | 0.3% | |
| Others | 1.1% | |
| Elemental Composition | | |
| Arsenic | Max 0.5 | ppm |
| Copper | Max 0.1 | ppm |
| Iron | Max 0.5 | ppm |
| Lead | Max 0.2 | ppm |
| Mercury | Max 0.04 | ppm |
| Chemical Characteristics | | |
| Peroxide value | Max 10.0 | meq/kg |

ND = not detectable

In some embodiments, an oil is characterized by one or more the following fatty acids (or esters thereof), expressed as % (wt/wt) of the total fatty acid content. The embodiments provided herein may further comprise about 12% or less, or about 6% to about 12% (wt/wt) of myristic acid (C14:0). The embodiments provided herein may further comprise about 28% or less, or about 18 to about 28% (wt/wt) of palmitic acid (C16:0). The embodiments provided herein may further comprise about 2% or less (wt/wt) of stearic acid (C18:0). The embodiments provided herein may further comprise about 8% or less of (wt/wt) oleic acid (C18:1n-9). The embodiments provided herein may further comprise about 2% or less (wt/wt) of linoleic acid (C18:2). The embodiments provided herein may further comprise about 2% or less (wt/wt) of arachidonic acid (C20:4). The embodiments provided herein may further comprise about 3% or less (wt/wt) of eicosapentaenoic acid (C20:5). The embodiments provided herein may further comprise about 18% or less, or about 12% to about 18% (wt/wt) of decosapentaenoic acid (22:5n-6). The embodiments provided herein may further comprise about 10% or less (wt/wt) of other fatty acids. In some of these embodiments, the ratio of % (wt/wt) of DHA to % (wt/wt) of DPAn6 is about 2.5 to about 2.7.

An oil with the preceding characteristics may comprise Life's DHA™ (also formerly referenced as DHA™-S and DHASCO®-S), Martek Biosciences, Columbia, Md.), an oil derived from the Thraustochytrid, Schizochytrium sp., that contains a high amount of DHA and also contains docosapentaenoic acid (n-6) (DPAn6). In some embodiments, more specifically, DHA™-S contains the following approximate exemplary amounts of these fatty acids, as a percentage of total fatty acids: myristic acid (14:0) 8.71%; palmitic acid (16:0) 22.15%; stearic acid (18:0) 0.66%; linoleic acid (18:2) 0.46%; arachidonic acid (20:4) 0.52%; eicosapentenoic acid (20:5, n-3) 1.36%; docosapentaenoic acid (22:5, n-6) (DPAn6) 16.28%; docosahexaenoic acid (DHA) (22:6, n-3) 41.14%; and others 8%.

In some embodiments, the dosage foam comprises, measured in percentage of free fatty acid, about 35-45% DHA (22:6 n-3); about 0-2% lauric acid (12:0); about 5-10% myristic acid (14:0); about 5-20% palmitic acid (16:0); about 0-5% palmitoleic acid (16:1); about 0-5% stearic acid (18:0); about 0-5% vaccenic acid or oleic acid (18:1n-7 and n-9, respectively); about 0-2% linoleic acid (18:2, n-6); about 0-5% stearidonic acid (18:4 n-3); about 0-10% 20:4 n-3, n-5, or n-6; about 0-2% adrenic acid 22:4 n-6; about 0-5% DPA n-3 (22:5); about 10-25% DPA n-6 (22:5); and 0-2% 24:0. In one embodiment, such an oil is derived from a microorganism of the genus Schizochytrium.

The DHA in an oil may be in the form of a DHA ester, preferably an alkyl ester, such as a methyl ester, ethyl ester, propyl ester, or combinations thereof, prepared from an algal oil derived from the Thraustochytrid, Schizochytrium sp. An exemplary DHA (ethyl esters) containing oil derived from Schizochytrium sp. is characterized by the specified amount of components listed in Table 4 of WO 2009/006317, incorporated by reference herein. In some of these embodiments, an oil comprises DHA greater than about 85% (wt/wt), particularly >about 86% (wt/wt) of the total fatty acid content of the oil or unit dose. In some of these embodiments, the ratio of % (wt/wt) of DHA to % (wt/wt) of DPAn6 is about 2.5 to about 2.7, or greater than 2.7.

In some embodiments, the composition or oil is characterized by one or more the following fatty acids (or esters thereof, particularly ethyl esters), expressed as % (wt/wt) of the total fatty acid content. The embodiments provided herein may further comprise about 0.5% or less (wt/wt) of lauric acid (C12:0). The embodiments provided herein may further comprise about 2% or less (wt/wt) of myristic acid (C14:0). The embodiments provided herein may further comprise about 0.5% or less (wt/wt) of myristoleic acid (C14:1). The embodiments provided herein may further comprise about 1% or less of palmitic acid (C16:0). The embodiments provided herein may further comprise about 1% or less (wt/wt) of linoleic acid (C18:2) (n-6). The embodiments provided herein may further comprise about 3% or less (wt/wt) of dihomo gamma linolenic acid (C20:3) (n-6). The embodiments provided herein may further comprise about 0.5% or less (wt/wt) of eicosatrienoic (C20:3) (n-3). The embodiments provided herein may further comprise about 1% or less (wt/wt) of arachidonic acid (C20:4). The embodiments provided herein may further comprise about 3% or less (wt/wt) of eicosapentaenoic acid (C20:5) (n-3). The embodiments provided herein may further comprise about 3% or less (wt/wt) of docosatrienoic acid (22:3). The embodiments provided herein may further comprise about 27% or less (wt/wt) of decosapentaenoic acid (22:5) (n-6). The embodiments provided herein may further comprise about 10% or less (wt/wt) of other components. In some of these embodiments, the ratio of % (wt/wt) of DHA to % (wt/wt) of DPAn6 is about 2.5 to about 2.7. An oil with the preceding characteristics may comprise ethyl ester oil derived from the oil of Thraustochytrid, Schizochytrium sp.

In some embodiments, the present invention further includes use of compositions comprising at least about 70% (wt/wt) DHA and at least about 15, 20, or 25% (wt/wt) DPAn6.

In some embodiments, the saturated fatty acid or an ester thereof may contain less than 20 carbons, such as, for example, a saturated fatty acid or an ester thereof that contains 19, 18, 17. 16, 15, 14, 13, 12, 11, 10, 9 or 8 carbons. In certain embodiments, the saturated fatty acid or ester thereof may contain 14 or 16 carbons.

In some embodiments, the composition of DHA may further comprise vitamin E. Compounds of the vitamin E group are fat-soluble vitamins with antioxidant properties and include eight related α-, β-, and γ-tocopherols and the corresponding four tocotrienols. In some embodiments, the vitamin E in the composition is a tocopherol. In some embodiments, the tocopherol is selected from α-, β-, and γ-tocopherols, or combinations thereof.

In the course of examination of a subject, a medical professional can determine that administration of DHA pursuant to one of the methods described herein is appropriate for the subject, or the physician can determine that the subject's condition can be improved by the administration of DHA pursuant to one of the methods described herein. Prior to prescribing any DHA regimen, the physician can counsel the subject, for example, on the various risks and benefits associated with the regimen. The subject can be provided full disclosure of all the known and suspected risks associated with the regimen. Such counseling can be provided verbally, as well as in written form. In some embodiments, the physician can provide the subject with literature materials on the regimen, such as product information, educational materials, and the like.

The present invention is also directed to methods of educating consumers about the methods of treating neurological disorders, the method comprising distributing the DHA dosage forms with consumer information at a point of sale. In some embodiments, the distribution will occur at a point of sale having a pharmacist or healthcare provider.

The term “consumer information” can include, but is not limited to, an English language text, non-English language text, visual image, chart, telephone recording, website, and access to a live customer service representative. In some embodiments, consumer information will provide directions for use of the DHA unit dosages according to the methods described herein, appropriate age, use, indication, contraindications, appropriate dosing, warnings, telephone number, and website address. In some embodiments, the method further comprises providing professional information to relevant persons in a position to answer consumer questions regarding use of the disclosed regimens according to the methods described herein. The term “professional information” includes, but is not limited to, information concerning the regimen when administered according to the methods of the present invention that is designed to enable a medical professional to answer customer questions.

A “medical professional,” includes, for example, a physician, physician assistant, nurse practitioner, pharmacist and customer service representative. All of the various aspects, embodiments and options described herein can be combined in any and all variations.

In some embodiments, the DHA is administered in a single dosage form, i.e., a dosage form, or in two or more dosage forms. As used herein, “dosage form” refers to the physical form for the route of administration. The term “dosage form” can refer to any traditionally used or medically accepted administrative forms, such as oral administrative forms, intravenous administrative forms, or intraperitoneal administrative forms. In some embodiments, the DHA is administered in a single dose, i.e., a unit dose. As used herein, a “unit dose” refers to an amount of DHA administered to a subject in a single dose, e.g., in a gel capsule. The term “unit dose” can also refer to a single unit of pharmaceutically suitable solid, liquid, syrup, beverage, or food item, that is administered within a short period of time, e.g., within about 1 minute, 2 minutes, 3 minutes, 5 minutes, 10 minutes, 20 minutes, or 30 minutes.

In some embodiments, the subject to be treated can be administered at least one unit dose per day. In some embodiments, the dosage forms can be taken in a single application or multiple applications per day. For example, if four capsules are taken daily, each capsule comprising about 500 mg DHA, then all four capsules could be taken once daily, or 2 capsules could be taken twice daily, or 1 capsule could be taken every 6 hours. Various amounts of DHA can be in a unit dose. In some embodiments, the unit dose comprises about 850 mg, about 900 mg, about 950 mg, about 1 g, or about 1.5 g, DHA.

In some embodiments, the dosage form has a total weight of about 0.2 g to about 2 g. By way of example and not limitation, a capsule can contain a total weight of an oil derived from an algal oil of about 0.85 g, about 0.9 g, about 0.95 g, about 1 g or about 1.05 g.

For the purposes herein, the composition of DHA may be administered daily and for a time period sufficient to provide a therapeutic benefit to the subject. As used herein, “daily dosage,” “daily dosage level,” “daily dosage amount” or “per day dosage” refer to the total amount of DHA (e.g., in the form of free fatty acids, alkyl esters, or triglycerides) administered per day (about 24 hour period). For example, administration of DHA to a subject at a dosage of 2 g per day means that the subject receives a total of 2 g of DHA on a daily basis, whether the DHA is administered as a single dosage form comprising 2 g DHA, or alternatively, four dosage forms comprising 500 mg DHA each (for a total of 2 g DHA). The composition of DHA may be taken in a single application or multiple applications per day. For example, if four capsules are taken daily, each capsule comprising 500 mg DHA, then all four capsules could be taken once daily, or 2 capsules could be taken twice daily, or 1 capsule could be taken every 6 hours. In some embodiments, the daily amount of DHA is administered at least once per day (e.g., single dosage form daily) or at least twice per day (e.g., in two or more dosage forms daily). In some embodiments, the DHA is administered at least two times weekly.

In some embodiments, the DHA is administered in an amount of from about 1.5 mg per kg body weight per day to about 125 mg per kg body weight per day. In some embodiments, the DHA is administered in an amount of from about 150 mg to about 10 g per day; from about 0.5 g per day to about 5 g per day; or from about 1 g per day to about 5 g per day.

In some embodiments, the daily amount of DHA administered comprises about 200 mg, 400 mg, 450 mg, 500 mg, 520 mg, 540 mg, 600 mg, 700 mg, 800 mg, 900 mg, 1 g, 1.5 g, 1.8 g, 2.0 g, 2.5 g, 2.7 g, 3.0 g, 3.2 g, 3.3 g, 3.4 g, 3.5 g, 3.6 g, 3.7 g, 3.8 g, 3.9 g, 4.0 g, 4.5 g, 5.0 g, 6.0 g, 6.5 g, 7 g, 8 g, 9 g, or 10 g DHA per day. In some embodiments, the DHA is administered in an amount of at least about 1 g per day.

In some embodiments, the daily dose of DHA administered to a human subject ranges from about 860 mg up to about 6 grams, particularly from about 1.7 grams up to about 6 grams, from about 2.6 grams up to about 6 grams, particularly from about 3.4 grams up to about 6 grams, particularly from about 4.3 grams to about 6 grams and more particularly from about 5.1 grams to about 6 gram. In some embodiments the daily dose of DHA administered to a human subject ranges from about 860 mg up to about 4 grams, particularly from about 1.7 grams up to about 4 grams, from about 2.6 grams up to about 4 grams, and more particularly from about 3.4 grams up to about 4 grams. In some embodiment the daily dose of DHA administered to a human subject ranges from about 860 mg up to about 1 gram, particularly from about 860 mg up to about 950 mg. In some embodiments, the daily dose of DHA administered ranges from about 1.7 grams up to about 2 grams, particularly from about 1.7 gram up to about 1.8 grams. In some embodiments, the daily dose of DHA administered to a human subject ranges from about 2.6 grams up to about 3 grams, particularly from about 2.6 grams up to about 2.8 grams. In some embodiments, the daily dose of DHA administered to a human subject is from about 3.4 grams up to about 4 grams, particularly from about 3.4 grams up to about 3.8 grams. In some embodiments, the daily dose of DHA administered to a human subject is from about 4.3 to about 5 grams, particularly from 4.3 grams to about 4.8 grams. In some embodiments, the daily dose of DHA administered to a human subject is from about 5.1 to about 6 grams, particularly from about 5.1 to about 5.7 grams.

In some embodiments, the daily dose is provided as a unit dose.

Various amounts of DHA may be in a dosage form. In some embodiments, the dosage form of DHA comprises about 900 mg DHA.

Administration of the DHA may be achieved using various regimens. For example, in some embodiments, administration of the DHA is daily on consecutive days, or alternatively, the dosage form is administered every other day (bi-daily). Administration may occur on one or more days. For example, in some embodiments the DHA is administered daily for the duration of the subject's lifetime, or from 1 year to 20 years or 5 years to 10 years. In some embodiments, administration of the DHA dosage form occurs for 7, 14, 21, or 28 days. In some embodiments, the DHA is administered for at least 6 months, for at least 1 yr, for at least 1.5 yrs., for at least 2 yrs., or for at least 5 yrs. In some embodiments, administration of the DHA occurs until a symptom of dementia or AD, e.g., loss of cognitive ability, is halted or reduced, the target being determined by a medical professional.

In some embodiments, the DHA is administered continuously. The term “continuous” or “consecutive,” as used herein in reference to “administration,” means that the frequency of administration is at least once daily. Note, however, that the frequency of administration can be greater than once daily and still be “continuous” or “consecutive,” e.g., twice or even three or four times daily, as long as the dosage levels as specified herein are achieved.

The term “administering” or “administration” of the composition refers to the application of the composition, e.g., oral or parenteral (e.g., transmucosal, intravenous, intramuscular, subcutaneous, rectal, intravaginal, or via inhalation) to the subject. Administering would also include the act of prescribing a composition described herein to a subject by a medical professional for treatment of AD. Administering can also include the act of labeling a composition, i.e., instructing a subject to administer a composition, in a manner as provided herein for treatment of AD. By way of example, administration may be by parenteral, subcutaneous, intravenous (bolus or infusion), intramuscular, or intraperitoneal routes. Dosage forms for these modes of administration may include conventional forms, either as liquid solutions or suspensions, solid forms suitable for solution or suspension in liquid prior to injection, or as emulsions.

Although fatty acids such as DHA can be administered topically or as an injectable, a preferred route of administration is oral administration. Preferably, the DHA composition is administered to individuals in the form of nutritional supplements, foods, pharmaceutical formulations, or beverages, particularly foods, beverages, or nutritional supplements, more particularly, foods and beverages, more particularly foods. A preferred type of food is a medical food (e.g., a food which is in a formulation to be consumed or administered externally under the supervision of a physician and which is intended for the specific dietary management of a disease or condition for which distinctive nutritional requirements, based on recognized scientific principles, are established by medical evaluation.).

In some embodiments, the dosage form is a pharmaceutical dosage form.

“Pharmaceutically acceptable” refers to compositions that are, within the scope of sound medical judgment, suitable for contact with the tissues of human beings and animals without excessive toxicity or other complications commensurate with a reasonable benefit/risk ratio. In some embodiments, the compounds (e.g., DHA), compositions, and dosage forms of the present invention are pharmaceutically acceptable.

The DHA can be formulated in a dosage form. These dosage forms can include, but are not limited to, tablets, capsules, cachets, pellets, pills, gelatin capsules, powders, and granules; and parenteral dosage forms which include, but are not limited to, solutions, suspensions, emulsions, coated particles, and dry powder comprising an effective amount of the DHA as taught in this invention. In some embodiments, the dosage form can be inserted or mixed into a food substance. Various substances are known in the art to coat particles, including cellulose derivatives, e.g., microcrystalline cellulose, methyl cellulose, carboxymethyl cellulose; polyalkylene glycol derivatives, e.g., polyethylene glycol; talc, starch, methacrylates, etc. In some embodiments, the dosage form is a capsule, wherein the capsule is filled with a solution, suspension, or emulsion comprising the DHA. It is also known in the art that the active ingredients can be contained in such formulations with pharmaceutically acceptable excipients such as diluents, fillers, disintegrants, binders, lubricants, surfactants, hydrophobic vehicles, water soluble vehicles, emulsifiers, buffers, humectants, moisturizers, solubilizers, preservatives, flavorants, taste-masking agents, sweeteners, and the like. Suitable excipients can include, e.g., vegetable oils (e.g., corn, soy, safflower, sunflower, or canola oil). In some embodiments, the preservative can be an antioxidant, e.g., sodium sulfite, potassium sulfite, metabisulfite, bisulfites, thiosulfates, thioglycerol, thiosorbitol, cysteine hydrochloride, α-tocopherol, and combinations thereof. The means and methods for administration are known in the art and an artisan can refer to various pharmacologic references for guidance. For example, “Modern Pharmaceutics,” Banker & Rhodes, Informa Healthcare, 4th ed. (2002); “Goodman & Gilman's The Pharmaceutical Basis of Therapeutics,” McGraw-Hill, New York, 10th ed. (2001); and Remingtons's Pharmaceutical Sciences, 20th Ed., 2001 can be consulted.

The DHA of the present invention is orally active and this route of administration can be used for the methods described herein. Accordingly, administration forms can include, but are not limited to, tablets, dragees, capsules, caplets, gelatin capsules, and pills, which contain the DHA and one or more suitable pharmaceutically acceptable carriers.

For oral administration, the DHA can be administered as an oil or it can be formulated readily by combining it with a pharmaceutically acceptable carrier or with pharmaceutically acceptable carriers. Pharmaceutical acceptable carriers are well known in the art. Such carriers enable the compounds of the invention to be formulated as tablets, gelatin capsules, pills, dragees, capsules, liquids, gels, syrups, slurries, suspensions and the like, for oral ingestion by a subject to be treated. In some embodiments, the dosage form is a tablet, gelatin capsule, pill or caplet. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl cellulose, sodium carboxymethyl cellulose, vegetable oil (e.g., soybean oil), and polyvinylpyrrolidone (PVP). If desired, disintegrating agents can be added, such as, but not limited to, the cross-linked polyvinyl pyrrolidone, agar, or alginic acid or a salt thereof such as sodium alginate. Pharmaceutical preparations which can be used orally include, but are not limited to, push-fit capsules made of gelatin, as well as soft, sealed capsules made of gelatin and a plasticizer, such as glycerol or sorbitol. Capsule shells can be composed of non-animal derived ingredients, i.e., vegetarian ingredients, such as carrageenan, alginate, modified forms of starch, cellulose and/or other polysaccharides. In specific embodiments, the gelatin capsules may be porcine, bovine, vegetarian, or alginate gelatin capsules. All formulations for oral administration should be in dosages suitable for such administration.

It should be understood that in addition to the ingredients particularly mentioned above, the formulations of this invention can include other suitable agents such as flavoring agents, preservatives, and antioxidants. In particular, it is desirable to mix the microbial oils with an antioxidant to prevent oxidation of the DHA. Such antioxidants are pharmaceutically acceptable and can include vitamin E, carotene, BHT or other antioxidants known to those of skill in the art.

In some embodiments, the dosage form is a nutraceutical dosage form. The term “nutraceutical” refers to any substance that is (1) a sole item of a meal or diet that provides medical and/or health benefits, or (2) a product that is intended to supplement the diet that bears or contains one or more of the following dietary ingredients: a vitamin, a mineral, an herb or other botanical, an amino acid, a dietary substance for use by man to supplement the diet by increasing the total daily intake, or a concentrate, metabolite, constituent, extract, or combinations of these ingredients that provides medical and/or health benefits. The medical and/or health benefits can include reducing the risk of a neurological disorder described herein.

In some embodiments, the DHA can be provided in a dietary supplement, medical food or animal feed. “Dietary supplement” refers to a compound or composition used to supplement the diet of an animal or human. In some embodiments, the dietary supplement can further comprise various “dietary ingredients” intended to supplement the diet. “Dietary ingredients” can further include: vitamins, minerals, herbs or other botanicals, amino acids, and substances such as enzymes, organ tissues, glandulars, and metabolites. Dietary ingredients can also include extracts or concentrates. In some embodiments, the dosage form of DHA is administered in a dietary supplement.

In some embodiments, the DHA is provided as a medical food for the dietary management of DHA levels in a human subject who is suffering from Alzheimer's disease, particularly one suffering mild to moderate AD. In some embodiments, the DHA is provided in an amount sufficient to increase the DHA levels in plasma phospholipid DHA of a subject who is ApoE4 negative and who is suffering from AD, particularly suffering from mild to moderate AD, more particularly from mild AD.

In some embodiments, DHA is provided as a medical food in an amount sufficient to increase the DHA levels in cerebrospinal fluid of a human subject suffering from AD, particularly mild to moderate AD, more particularly with mild AD.

The present invention is also directed to use of an oral dosage form consisting essentially of about 850 to about 940 mg of docosahexaenoic acid (DHA) wherein the dosage form comprises less than about 1% eicosapentaenoic acid (EPA) and less than about 2% docosapentaenoic acid 22:5n-6 (DPAn6). In some embodiments, the oral dosage form is a unit dosage form, in particular, a gelatin capsule. Optionally the gelatin capsule also comprises a colorant, flavoring, and/or antioxidant.

The present invention is directed to methods of lowering plasma triglyceride levels. In some embodiments, the methods also result in a lowering of the amount of total cholesterol in the subject. The measurement of plasma triglyceride levels and total cholesterol can be accomplished using any of the commercially available devices, e.g., a Vitros 750 (Johnson & Johnson, Ortho Clinical Diagnostics, Raritan, N.J.), or an Olympus AU640™ Chemistry Immune Analyzer (Center Valley, Pa.). The term “lowering” refers to the reduction of plasma triglyceride levels in a subject, wherein the triglyceride levels of the subject are measured before and after administration of the DHA ester dosage form. As one of skill in the art will recognize, triglyceride levels fluctuate in a subject depending on many factors, e.g., diet (e.g., fasting conditions), exercise regimens, time of day, etc. Thus, the term “lowering” refers to the relative amounts of triglyceride levels of a subject before and after administration of DHA ester, wherein the diet, exercise regimens and time of day are controlled. Similarly, the term “lowering” total cholesterol refers to the reduction of total cholesterol in a subject, wherein the total cholesterol levels of the subject are measured before and after administration of the DHA ester dosage form. As one of skill in the art will recognize, total cholesterol levels fluctuate in a subject depending on many factors, e.g., diet (e.g., fasting conditions), exercise regimens, time of day, etc. Thus, the term “lowering” refers to the relative total cholesterol levels of a subject before and after administration of DHA ester, wherein the diet, exercise regimens and time of day are controlled.

The triglyceride levels in a subject can be reduced relative to a subject that has not been administered a dosage form comprising DHA ester. In some embodiments of the present invention, the triglyceride levels are reduced greater than 5%, or about 5% to about 90%, about 10% to about 80%, about 25% to about 75%, or about 30% to about 65%. In some instances, the triglyceride levels in a subject can be reduced relative to a subject that has been administered a dosage form comprising DHA and EPA, e.g., Lovaza®. In these instances, the triglyceride levels can be reduced greater than 5%, or about 5% to about 90%, about 10% to about 80%, about 25% to about 75%, or about 30% to about 65% relative to a subject administered DHA and EPA. One of skill in the art will appreciate that the amount of the reduction can be dependent on the initial triglyceride level in the subject. For example, in subjects having a higher original triglyceride level, the amount of triglyceride reduction can be greater, relative to a subject with a lower original triglyceride level. Triglyceride levels reduction can also be dependent on the length and/or amount of administration of DHA ester, or the regimen of administration of the DHA ester. For example, in some embodiments, the subject has a chronic condition, and is administered the DHA ester of the present invention for the remainder of the subject's lifetime, or from 1 to 20 years, or 1, 2, 5, 10, or 15 years. In some embodiments, the triglyceride levels in a subject are reduced by greater than 5%, about 5% to about 90%, about 25% to about 75%, or about 30% to about 65% by 1, 5, 10, 15 or 20 years. In certain embodiments, the triglyceride level in a subject is reduced by a percentage that is within a range, inclusive or exclusive of endpoints, wherein the upper and lower limits of the range are independently selected from the following amounts: about 5%, about 10%, about 15%, about 20%, about 25%, about 30%, about 35%, about 40%, about 50%; about 60%, about 70%, about 80% and about 90%; optionally within a time period selected from the following times periods: about 7 days, about 2 weeks, about 1 month, about 6 weeks, about 2 months, about 3 months, about 6 months, about 1 year, and about 5 years.

In those embodiments that also result in a lowering of the amount of total cholesterol in the subject, the amount of total cholesterol in the subject will be reduced relative to a subject that has not been administered a dosage form comprising DHA ester. In some embodiments of the present invention, the total cholesterol levels are reduced by about 15%, about 20%, about 25%, about 40%, about 15% to about 25%, or about 20% to about 40%. In certain embodiments in which the subject's total cholesterol is lowered, the total cholesterol level in a subject is reduced by a percentage that is within a range, inclusive or exclusive of endpoints, wherein the upper and lower limits of the range are independently selected from the following amounts: about 10%, about 15%, about 20%, about 25%, about 30%, about 35%, about 40%, and about 50%; optionally within a time period selected from the following times periods: about 7 days, about 2 weeks, about 1 month, about 6 weeks, about 2 months, about 3 months, about 6 months, about 1 year, and about 5 years. In some instances, the total cholesterol levels in a subject can be reduced relative to a subject that has been administered a dosage form comprising DHA and EPA, e.g., Lovaza®. One of skill in the art will appreciate that the amount of the reduction can be dependent on the initial total cholesterol level in the subject. For example, in subjects having a higher original total cholesterol levels, the amount of total cholesterol reduction can be greater, relative to a subject with a lower original total cholesterol level. Total cholesterol level reduction can also be dependent on the length and/or amount of administration of DHA ester, or the regimen of administration of the DHA ester. For example, in some embodiments, the subject has a chronic condition, and is administered the DHA ester of the present invention for the remainder of the subject's lifetime, or from 1 to 20 years, or 1, 2, 5, 10, or 15 years.

In some embodiments, DHA ester of the present invention is administered daily for a shorter duration, e.g., 1 week to 12 weeks (week 1 to week 12). In some embodiments, the triglyceride levels in a subject are reduced by greater than 5%, about 5% to about 90%, about 25% to about 75%, or about 30% to about 65% on week 12. In those embodiments that also result in a lowering of the amount of total cholesterol in the subject, the total cholesterol levels in the subject are also reduced by about 15%, about 20%, about 25%, about 40%, about 15% to about 25%, or about 20% to about 40% on week 12. In some embodiments, the DHA ester is administered daily for 1 week to 6 weeks (week 1 to week 6). In some embodiments, the triglyceride levels in a subject are reduced by greater than 5%, about 5% to about 90%, about 25% to about 75%, or about 30% to about 65% on week 6. In those embodiments that also result in a lowering of the amount of total cholesterol in the subject, the total cholesterol levels in a subject are also reduced by about 15%, about 20%, about 25%, about 40%, about 15% to about 25%, or about 20% to about 40% on week 6. In some embodiments, the DHA ester is administered daily for 2 weeks to 4 weeks (week 2 to week 6). In some embodiments, the triglyceride levels in a subject are reduced by greater than 5%, about 5% to about 90%, about 25% to about 75%, or about 30% to about 65% on week 6. In some embodiments, the total cholesterol levels in a subject are also reduced by about 15%, about 20%, about 25%, about 40%, about 15% to about 25%, or about 20% to about 40% on week 6. In some embodiments, the DHA ester is administered daily for 28 days (day 28). In some embodiments, the triglyceride levels in a subject are reduced by greater than 5%, about 5% to about 90%, about 25% to about 75%, or about 30% to about 65% by day 28. In some embodiments, the total cholesterol levels in a subject are also reduced by about 15%, about 20%, about 25%, about 40%, about 15% to about 25%, or about 20% to about 40% by day 28. In some embodiments, the DHA ester is administered daily for 14 days (day 14). In some embodiments, the triglyceride levels in a subject are reduced by greater than 5%, about 5% to about 90%, about 25% to about 75%, or about 30% to about 65% by day 14. In some embodiments, the total cholesterol levels in a subject are also reduced by about 15%, about 20%, about 25%, about 40%, about 15% to about 25%, or about 20% to about 40% by day 14. In some embodiments, the DHA ester is administered daily for 7 days (day 7). In some embodiments, the triglyceride levels in a subject are reduced greater than 5%, about 5% to about 90%, about 25% to about 75%, or about 30% to about 65% by day 7. In some embodiments, the total cholesterol levels in a subject are also reduced by about 15%, about 20%, about 25%, about 40%, about 15% to about 25%, or about 20% to about 40% by day 7.

In some embodiments of the present invention, the DHA ester provides a rapid onset of triglyceride level reduction, relative to administration of (a) a composition comprising DHA and EPA, (b) triglyceride form of DHA, and/or (c) an impure form of DHA (e.g., a composition wherein <79% of the total fatty acid composition is DHA). In some embodiments, the impure form of DHA comprises a composition wherein <60%, <70%, <80%, <85%, <90%, <95%, <96%, <97%, <98%, <99%, <99.5%, <99.8% or <99.9% of the total fatty acid composition is DHA. The term “rapid onset” refers to the reduced time needed to lower a subject's triglyceride level to a designated point. For example, in some embodiments, the DHA ester of the present invention reduces the triglyceride level 5%, 10%, 15%, 20%, 30%, 40%, or 50% faster than a composition comprising (a) DHA and EPA, (b) triglyceride form of DHA, and/or (c) an impure form of DHA (e.g., a composition wherein <79% of the total fatty acid composition is DHA). Similarly, in certain embodiments that also result in a lowering of the amount of total cholesterol in the subject, the DHA ester provides a rapid onset of total cholesterol reduction.

The method of the present invention can be administered to individuals who have normal triglyceride levels (under 150 mg triglyceride/dL), or elevated triglyceride levels, e.g., borderline high triglyceride levels (151-200 mg triglyceride/dL), high triglyceride levels (201-499 mg triglyceride/dL), or very high triglyceride levels (hypertriglyceridemia) (>500 mg triglyceride/dL). Thus, in some embodiments the invention is directed to a method of treating a subject having normal triglyceride levels, borderline high triglyceride levels, high triglyceride levels, or very high triglyceride levels, the method comprising administration of the DHA esters as described herein. In some embodiments, the DHA esters as described herein are administered as an adjunct to diet to reduce triglyceride levels. In certain embodiments, the subject is an adult subject with very high triglyceride levels, i.e., hypertriglyceridemia. In some embodiments, the present invention is directed to methods of treating hypertriglyceridemia (elevated triglyceride levels), comprising administration of DHA esters as described herein. Hypertriglyceridemia can include familial hypertriglyceridemia. In some embodiments, the method of the present invention can be used to treat chronic elevated (i.e., borderline high triglyceride levels, high triglyceride levels, or very high triglyceride levels) triglyceride levels for the remainder of the life of the subject.

In some embodiments, the subject has normal total cholesterol levels (under 200 mg/dL), or elevated total cholesterol, e.g., borderline total cholesterol levels (200-239 mg/dL) or high total cholesterol levels (240 mg/dL or greater). In some embodiments, the methods of the invention result in a lowering of a subject's total cholesterol from high total cholesterol to borderline or normal total cholesterol levels. In some embodiments, the methods of the invention result in a lowering of a subject's total cholesterol from borderline to normal total cholesterol.

In some embodiments, the dosage form of the present invention can be used in the treatment of Alzheimer's disease in patient that are ApoE 4 negative as described in U.S. application Ser. No. 12/833,913, incorporated herein by reference in its entirety. In some embodiments, the dosage form of the present invention can be used in treatment of mild cognitive impairment and/or age related cognitive decline, as described in U.S. application Ser. Nos. 11/483,138 and 12/699,009, incorporated herein by reference in their entirety. In some embodiments, the dosage form of the present invention can be used in the lowering of triglyceride levels, as described in U.S. application Ser. No. 12/572,263, incorporated herein by reference in its entirety.

The oral dosage form of the invention optionally includes an antioxidant. The use of any antioxidant added to foods or pharmaceuticals is encompassed by the invention. For example, antioxidants that are optionally contained in the oral dosage form of the invention include, but are not limited to, ascorbyl palmitate, catechin, tocopherol, vitamin C fatty acid esters, butylated hydroxytoluene (BHT), butylated hydroxyanisole (BHA), tertiary butylated hydroquinone (TBHQ), phospholipids, natural antioxidants, and combinations thereof. Appropriate antioxidant concentrations in the oral dosage form can readily be determined using known techniques for evaluating chemical stability. In some embodiments, the amount of the optional antioxidant is from about 0.01 to 5% (wt/wt) or from 0.05 to 1% (w//w) of the weight of the liquid contents of the dosage form.

In one embodiment, the gel cap is a hard or a soft-gelatin capsule made from gelatin, glycerol and water, and filled with DHA-EE and an antioxidant. In some embodiments, the encapsulating material comprises a gelatin, a plasticizer, and water. Plasticizers of the invention include glycerin, glycerol, polyols, and mixtures thereof. In some embodiments, the plasticizer is a high boiling point polyol, such as glycerol or sorbitol. In certain embodiments, the gel cap is animal or vegetable derived. In one embodiment the gel cap is filled with DHA ester, preferably DHA-EE. In one embodiment, the gel cap comprises a 1 gram dosage form, wherein the fill weight of the dosage form is from about 950 to about 1050 mg, and wherein the gel cap contains from about 855 mg/g to about 945 mg/g DHA-ethyl ester. In some embodiments, the gelatin capsule comprises a 1 gram dosage form, wherein the fill weight of the dosage form is from about 950 mg to about 1050 mg, and wherein the gelatin capsule comprises from about 860 mg to about 950 mg DHA per 1000 mg of the dosage form. In some embodiments, the gelatin capsule comprises about 900 mg DHA per 1,000 g of the dosage form.

In certain embodiments, the DHA esters of the invention comprise about 90%, about 91%, about 92%, from about 85.5% to about 94.5%, from about 90 to about 92%, or from about 87.5% to about 92.5% wt of the total fatty acid content of the dosage form. In one embodiment, the DHA esters of the invention comprise greater than, or greater than or equal to, about 90%, about 91%, or about 92% wt of the total fatty acid content of the dosage. Preferably the DHA esters of the invention comprise about 90% wt of the total fatty acid content of the dosage. In a one gram dosage form embodiment, the gel cap contains about 900 mg DHA per 1,000 g of the dosage. In one embodiment, the gel cap contains between about 900 mg to about 1 gram of DHA ester. In a preferred embodiment, the gel caps of the invention are administered in an amount up to a daily amount of 2 grams of DHA ester. In one embodiment, the DHA ester of the invention is derived from marine microalgae, preferably C. cohnii. In certain aspects of the invention, the gel caps are administered in daily dosage amounts, and regimens as described herein. In certain aspects of the invention, the gel caps are formulated in dosage amounts as described herein.

In certain embodiments, the encapsulating material is vegetarian, i.e., made from non-animal derived material, including plants, seaweed (for example, carrageenan), food starch, modified corn starch, potato starch, and tapioca. In some embodiments, the gelatin use in the gel cap formulation is bovine gelatin, porcine gelatin, fish gelatin, or a mixture thereof.

In certain embodiments, the gel cap is colored. Any of a variety of colorants known in the industry may be utilized including, for example, natural or synthetic derivatives, or combinations thereof. In particular embodiments, the type and intensity of color is designed to enhance consumer acceptance. In other embodiments, the type and intensity of color is chosen so as to reduce DHA photo-oxidation. In some embodiments, the gel cap is clear or semi-transparent. In other embodiments the gel cap is semi-opaque.

In certain embodiments, the gelatin capsule is vegetarian. In some embodiments, the capsule preparation comprises no animal products, and comprises glycerol (and/or other polyols), seaweed extract (carrageenan) and water. In some embodiments, the water is purified. In some embodiments, color, flavor and/or sweeteners are added. During encapsulation, in some embodiments, fractionated coconut oil is used as a lubricant.

In some embodiments, the gelatin capsule comprises a capsule preparation, an active, and optionally a colorant and/or antioxidant. In another embodiment i) the capsule preparation comprises gelatin (bovine acid hide), glycerin, and purified water, ii) the active comprises DHA-EE, iii) the optional colorant is selected from titanium dioxide, FD&C Yellow #5, FD&C Red 40, and mixtures thereof; and iv) the antioxidant is ascorbyl palmitate. In some embodiments, the raw materials are USP raw materials.

In some embodiments, the gelatin capsules are soft gelatin capsules of about 1 g, having the specifications within the limits set forth in Table 3:

TABLE 3
Specifications for 1 gram DHA Ethyl Ester Gelatin Capsules

| TEST | METHOD | SPECIFICATION |
|---|---|---|
| Appearance | PDS-LAB-0104 | Orange opaque oblong |
| | | softgel, unprinted |
| Fill Appearance | PDS-LAB-0104 | Clear colorless to light |
| | | yellowish liquid |
| Identification | ATM-MBB-J0001 | Retention time of DHA |
| by GC | | ethyl ester peak in the |
| | | sample solution injection |
| | | corresponds to that in |
| | | the standard solution |
| | | injection over the run |
| Assay of | ATM-MBB-J0001 | 90.0-110.0% label claim |
| DHA EE | | |
| EPA | ATM-MBB-J0001 | |
| Peroxide | Current | Not more than 10 meq/Kg |
| Value | EP<2.5.1> | |
| Anisidine | Current | Not more than 20 |
| Value | EP<2.5.1> | |
| Acid Value | Current | Not more than 2.0 mg |
| | EP<2.5.1> | KOH/g |
| Uniformity of | Current USP | Conforms to Current USP |
| Dosage Units | <905> | for Weight Variation of |
| | | Soft Capsules |
| Disintegration | Current USP | |
| | <701> | |

Microbiological Testing: harmonized USP, Ph. Eur., JP

| | | |
|---|---|---|
| Total aerobic | PDS-MIC-0031 | ≦1000 cfu/g |
| micorbial count | | |
| (total plate count) | | |
| Total combined mold | PDS-MIC-0031 | ≦100 cfu/g |
| & yeast count | | |
| Escherichia coli | PDS-MIC-0031 | Absent in 1 g |
| Almonella species | PDS-MIC-0031 | Absent in 10 g |
| Staphylococcus aureus | PDS-MIC-0031 | Absent in 1 g |
| Pseudomonas | PDS-MIC-0031 | Absent in 1 g |
| aeruginosa | | |

Set forth in Table 4 is a list of components that are, in some embodiments, used in the manufacture of a DHA-EE soft gelatin capsule, and at least one corresponding function for each component.

TABLE 4
List of Components in 1 gram DHA
Ethyl Ester Soft Gelatin Capsules

| Component | Function |
|---|---|
| 900 mg DHA EE | Active |
| Gelatin, Bovine Acid Hide | Capsule Preparation |
| Glycerin | Capsule Preparation |
| Purified Water | Capsule Preparation |
| Titanium Dioxide | Colorant |
| FD&C Yellow #5 | Colorant |
| FD&C Red #40 | Colorant |

Administration of DHA esters according to the methods described herein can achieve a pharmacokinetic profile of DHA similar to that of a composition comprising DHA and EPA, e.g., Lovaza® (Reliant Pharmaceuticals), even though DHA ester of the present invention is substantially free of EPA. For example, absorption, incorporation into membranes, hydrolysis by esterases, absorption in the enterocytes, introduction into chylomicrons, very low density lipoproteins (VLDL), low density lipoproteins (LDL), and high density lipoproteins (HDL) of the DHA esters can be similar to that observed with a composition comprising DHA and EPA. In some embodiments, absorption, incorporation into membranes, hydrolysis by esterases, absorption in the enterocytes, introduction into chylomicrons, very low density lipoproteins (VLDL), low density lipoproteins (LDL), and high density lipoproteins (HDL) of the DHA esters can occur more rapidly relative to that observed with a composition comprising (a) DHA and EPA, (b) triglyceride form of DHA, and/or (c) an impure form of DHA (e.g., a composition wherein <79% of the total fatty acid composition is DHA). In some embodiments, the DHA ester is absorbed, incorporated into membranes, or hydrolyzed, absorbed into enterocytes, and/or introduction into chylomicrons, VLDL, LDL, and/or HDL at a rate 5%, 10%, 15% or 20% faster than that observed with a composition comprising (a) DHA and EPA, (b) triglyceride form of DHA, an/or (c) an impure form of DHA. In some embodiments, the DHA esters according to the methods described herein can achieve a reduction in triglyceride levels in a subject similar to that of a composition comprising DHA and EPA.

Retroconversion is an enzymatic process during which long-chain fatty acids are converted to their related shorter-chain precursor fatty acids though the incremental removal of two-carbon units from the molecule. DHA can be retroconverted to EPA and DPAn-3. See, e.g., Brossard et al., Am. J. Clin. Nutr. 64:577-86 (1996). In some embodiments, the DHA ester of the present invention is retroconverted to a lesser degree (or at a reduced rate) relative to DHA free acid and/or a salt form, or a DHA triglyceride form. For example, in some embodiments, less EPA and/or DPAn-3 is produced in the method using DHA esters of the present invention, relative to a method using a DHA free acid and/or salt form, or a DHA triglyceride form.

Metabolism of DHA esters can also result in the formation of Resolvin D1, Resolvin D2, Resolvin D3, and Resolvin D4. See, e.g., Serhan et al., Annu. Rev. Pathol. Mech Dis. 3:279-312 (2008). In some embodiments, the DHA ester metabolites have a similar pharmacokinetic profile to the DHA esters in, e.g., Lovaza®, even though DHA ester of the present invention is substantially free of EPA.

Administration of the DHA ester dosage forms of the present invention can be achieved using various regimens. For example, in some embodiments, administration of the DHA ester dosage forms is daily on consecutive days, or alternatively, the dosage form is administered every other day (bi-daily). Administration can occur on one or more days. For example, in some embodiments the DHA ester is administered daily for the duration of the subject's lifetime, or from 1 year to 20 years or 5 years to 10 years. In some embodiments, administration of the DHA ester dosage form occurs for 7, 14, 21, or 28 days. In some embodiments, administration of the DHA ester dosage form occurs until the triglyceride levels of the subject are lowered to a preselected target level, the target level being determined by a medical professional. In some embodiments, administration of the DHA ester dosage form continues even after the triglyceride levels of the subject have reached normal or borderline levels, or to a preselected target level. In some embodiments, the administration of the DHA ester is administered as a prophylactic measure, before the triglyceride levels become elevated.

Administration of DHA ester dosage forms can be combined with other regimens (i.e., non-DHA ester regimens) used to reduce triglyceride levels. For example, the method of the present invention can be combined with diet regimens (e.g., low carbohydrate diets, high protein diets, high fiber diets, etc.), exercise regimens, weight loss regimens, or smoking cessation regimens to lower triglyceride levels. The methods of the present invention can also be used in combination with other pharmaceutical products to lower triglyceride levels in a subject. Non-DHA ester regimens can also include other triglyceride-lowering pharmaceutical products including, e.g., bile acid binding resins, e.g., cholestyramine and cholestipol; niacin; fibric acid derivatives, e.g., gemfibozil and clofibrate; and statins, e.g., lovastatin, pravastatin, atorvastatin and simvastatin.

In some embodiments, the DHA esters of the present invention are administered before the non-DHA ester regimens. For example, the DHA ester dosage forms can be first used to reduce triglyceride levels, followed by administration of the non-DHA ester regimens to maintain (or further lower) the preselected triglyceride level. Alternatively, in some embodiments, the non-DHA ester regimens are administered first to lower the triglyceride levels in a subject to a preselected target level, and then the DHA ester dosage forms of the present invention are administered to maintain (or further lower) the lowered triglyceride levels in the subject. Thus, in some embodiments, the present invention is directed to a method of maintaining triglyceride levels using the DHA ester dosage forms of the present invention, the method comprising (1) administering a non-DHA ester regimen to a subject to lower the triglyceride levels in the subject, until the triglyceride levels have reached a preselected triglyceride level, and (2) administering the DHA ester dosage foams of the present invention to maintain the preselected triglyceride level. In some embodiments, the preselected triglyceride level is a triglyceride level in the normal triglyceride level range or the borderline high triglyceride level range.

The present invention is directed to kits or packages containing one or more dosage forms to be administered according to the methods of the present invention. A kit or package can contain one dosage form, or more than one dosage forms (i.e., multiple dosage forms). If multiple dosage forms are present in the kit or package, the multiple dosage forms can be optionally arranged for sequential administration. In some embodiments, the dosage forms are packaged in blister cards or blister packs. The kits can contain dosage forms of a sufficient number to provide convenient administration to a subject who has a chronic condition and requires long-term administration of the DHA ester of the present invention. Each dosage form can contain about ≧850 DHA ester and can be intended for ingestion on successive days. For example, in some embodiments, the kit provides dosage forms of a sufficient number for 1, 2, 3 or 4 months of daily administration of the DHA ester. In some embodiments of the present invention, the kit comprises dosage forms for shorter periods of administration, e.g., the kit can contain about 7, 14, 21, 28 or more dosage forms for oral administration, each dosage form containing about ≧850 mg DHA ester and intended for ingestion on successive days. The method of the present invention can include administration of the dosage form daily for extended periods of time, e.g., 6 months, 1 year, 18 months, 2 years, 5 years, 10 years, 20 years, or indefinitely for the duration of a subject's life. The method also can include administration of the dosage form daily for shorter periods of time, e.g., once daily for at least 7, 14, 21, or 28 consecutive days. In some embodiments, the invention is directed to a method of reducing plasma triglyceride level in a subject, the method comprising administering daily to the subject a dosage form comprising about ≧850 of DHA ester substantially free of EPA, wherein the dosage form is administered daily for 4 to 28 consecutive days, or for 7 to 14 consecutive days.

The kits of the present invention can optionally contain instructions associated with the dosage fauns of the kits. Such instructions can be in a form prescribed by a governmental agency regulating the manufacture, use or sale of pharmaceutical products, which notice reflects approval by the agency of the manufacture, use or sale for human administration to treat a condition or disorder. The instructions can be in any form which conveys information on the use of the dosage forms in the kit according to the methods of the invention. For example, the instructions can be in the form of printed matter, or in the form of a pre-recorded media device.

### EXAMPLES

### Example 1

### Purification of DHA Ethyl Ester from Algal Source

This example illustrates a method for purifying ethyl docosahexaenoate from docosahexaenoic acid-containing single cell oil.

150 mL of absolute ethanol (EtOH) was added to 175 g (approximately 0.2 moles of triglyceride) of DHASCO®-T oil (Martek Biosciences Corporation, Columbia, Md., having a DHA content of 0.4 g/g oil) in a one-liter flask under nitrogen (N₂) at room temperature. DHASCO®-T oil is prepared from the microalgae Crypthecodinium cohnii. The mixture was allowed to stir for 15 minutes to obtain a homogeneous solution. Then 67 g of a 21% solution of sodium ethoxide/ethanol (NaOEt/EtOH; approximately 1.04 molar equivalents of triglycerides) was added to the solution and the mixture was allowed to reflux under N₂ for about 9 hours. The progress of the reaction was monitored by gas chromatography (GC) and thin-layer chromatography (TLC). When the reaction was completed, approximately 75 mL of EtOH was removed by distillation. The reaction mixture was then allowed to cool to room temperature under N₂. Hexane (300 mL) was added to the cooled reaction mixture, and the mixture was allowed to stir for 15 minutes at room temperature. Then 300 mL of deionized water was added to the mixture, and the mixture was allowed to stir for an additional 15 minutes. After removing and saving the organic layer, the aqueous layer was washed twice with 300 mL portions of hexane. A dark brown aqueous layer was discarded. The combined organic layers were then washed with 200 mL of a saturated NaCl solution. A GC analysis of the organic layer indicated the presence of about 44.7% DHA ethyl ester; the remaining materials were predominantly lower molecular weight ethyl esters (see Table 5).

The combined organic layer was concentrated under reduced pressure. The crude concentrate was then subjected to vacuum fractional distillation. The lower molecular weight ethyl esters were collected at temperatures between 100-150° C. and at a pressure of 0.8 mm Hg. The major components of this fraction were oleic, saturated C-14, and C-12 esters. The DHA ethyl ester was collected at temperatures between 155-165° C. and at a pressure of 0.8 mm Hg. A GC analysis of the DHA ethyl ester fraction showed a purity of about 91.3% DHA ethyl ester (see Table 5). From the fractional distillation, 68 g (86% yield) of the DHA ethyl ester was obtained as a light yellow oil.

TABLE 5
GC Analysis of DHASCO ®-T Oil Transesterification
and Distillation Products

| | | DHA Ethyl Ester-Containing |
|---|---|---|
| | Organic Layer After | Fraction After Vacuum |
| Sample | Transesterification | Fractional Distillation |
| %22:6 (n-3) DHA | 44.72 | 91.29 |
| %20:5 (n-3) EPA | 0.00 | 0.00 |
| % Additional | 55.28 | 8.81 |
| components | | |

### Example 2

### Purification of DHA Ethyl Ester

This example illustrates a method for purifying ethyl docosahexaneoate from a crude Crypthecodinium cohnii oil.

A crude oil obtained from Crypthecodinium cohnii by hexane extraction (DHA content of 0.5 g/g oil) was used directly without any further processing, such as winterization and/or RBD processing. Absolute ethanol (150 mL) was added to 175 g (approximately 0.2 moles of triglycerides) of the crude oil in a one-liter flask under N₂ at room temperature. The mixture was allowed to stir for 15 minutes to obtain a homogeneous solution. Then 67 g of a 21% solution of NaOEt/EtOH (approximately 1.04 molar equivalents of triglycerides) was added to the solution, and the mixture was allowed to reflux under N₂ for about 10 hours. The progress of the reaction was monitored by GC and TLC. When the reaction was completed, approximately 75 mL of ethanol was removed by distillation, and the mixture was allowed to cool to room temperature under N₂. Hexane (300 mL) was added to the cooled mixture, and the mixture was allowed to stir for 15 minutes at room temperature. 300 mL of deionized water was then added to the mixture, and the mixture was allowed to stir for an additional 15 minutes. After removing and saving the organic layer, the aqueous layer was washed twice with 300 mL portions of hexane. The combined organic layer was then washed with 200 mL of a saturated NaCl solution. A GC analysis of the organic layer indicated the presence of about 51% DHA ethyl ester; the remaining materials were predominantly lower molecular weight ethyl esters (see Table 6).

The combined organic layer was concentrated under reduced pressure. The crude concentrate was then subjected to vacuum fractional distillation. The lower molecular weight ethyl esters were collected at temperatures between 100-150° C. and at a pressure of 0.8 mm Hg. The major components of this fraction were oleic, saturated C-14, and C-12 esters. The DHA ethyl ester was collected at temperatures between 155-165° C. and at a pressure of 0.8 mm Hg. A GC analysis of the DHA ethyl ester fraction showed a purity of about 92% DHA (see Table 6). From the fractional distillation, 69 g (66% yield) of the DHA ethyl ester was obtained as a light yellow oil.

TABLE 6
GC Analysis of Crude Crypthecodinium cohnii Oil
Transesterification and Distillation Products

| | | DHA Ethyl Ester-Containing |
|---|---|---|
| | Organic Layer After | Fraction After Vacuum |
| Sample | Transesterification | Fractional Distillation |
| %22:6 (n-3) DHA | 51.25 | 91.80 |
| %20:5 (n-3) EPA | 0.00 | 0.00 |
| % Additional | 48.75 | 8.20 |
| components | | |

### Example 3

### Effect of DHA Ethyl Ester on Triglyceride Levels

The effect of the DHA ethyl ester (DHA-EE) produced according to the method of Example 2 on triglyceride levels was investigated using male Wister rats. The DHA-EE used in this example contained DHA at about 93.6% (wt/wt) of the total fatty acid content of the dosage form. The effect of purified DHA-EE was compared to two different DHA-containing products: DHASCO-T® (Martek Bioscience Corporation, Columbia, Md.) and Lovaza® (Reliant Pharmaceuticals, Inc., Durham, N.C.). DHASCO-T® comprises approximately 45% DHA and 55% other fatty acids (with substantially no EPA). Lovaza® comprises approximately 41.7% DHA ethyl ester, 51.7% EPA ethyl ester, and 6.4% other fatty acids. The vehicle control comprised corn oil. 84 Wistar rats were randomized into 7 groups of 12 rats each. Each rat was administered orally a high fructose diet for 4-5 weeks to raise triglyceride levels. After 4-5 weeks, rats with a triglyceride level <300 mg/dL were excluded. Then each rat was administered either (a) a vehicle control, (b) DHA ethyl ester (0.6 g/kg/day, 1.3 g/kg/day, 2.5 g/kg/day, or 5.0 g/kg/day), (c) DHASCO-T (5.0 g/kg/day), or (d) Lovaza (5.0 g/kg/day) for 28 days by oral gavage. Diets were controlled for calories, nutrients and levels of vitamin E. Triglyceride levels of the rats were measured at 14 days and 28 days. The results are presented in Table 7. Triglyceride values shown are averages for each treatment group, presented as actual mean values.

TABLE 7
Effect of DHA-EE, DHASCO-T, and Lovaza on Mean Triglyceride Levels^{a}

| | | Control | DHA-EE | | | | DHASCO-T ® | LOVAZA ® |
|---|---|---|---|---|---|---|---|---|
| | g/kg/day fatty acid | 0.0 | 0.6 | 1.3 | 2.5 | 5.0 | 5.0 | 5.0 |
| | g/kg/day DHA (approx) | 0.0 | 0.56 | 1.2 | 2.3 | 4.7 | 2.25 | 2.09 |
| 14 | Triglycerides mg/dL | 263.6 ± 100.3 | 176.5 ± 96.4^{†} | 175.2 ± 50.6^{†} | 115.5 ± 60.9^{†} | 97.6 ± 47.8^{†} | 120.8 ± 36.8^{†} | 125.2 ± 73.8^{†} |
| day | % of control | 100 | 67 | 66 | 44 | 37 | 46 | 47 |
| 28 | Triglycerides mg/dL | 192.2 ± 65.4 | 182.7 ± 86.9^{‡} | 114.7 ± 37.2^{†} | 107.9 ± 58.4^{†} | 78.9 ± 46.5^{†} | 122.6 ± 57.2 | 70.0 ± 28.9^{†} |
| day | % of control | 100 | 95 | 60 | 56 | 41 | 64 | 36 |

^{a}Data are presented as mean ± SD.
^{†}P < 0.05 vs control.
^{‡}P < 0.05 vs Lovaza.

The triglyceride results presented below in Table 8 are calculated from the same data set as in Table 7. Triglyceride values shown in Table 8 are averages for each treatment group, presented as least squares mean values.

TABLE 8
Effect of DHA-EE, DHASCO, and Lovaza on LSMEAN Serum Triglyceride Levels^{a}

| | | Control | DHA-EE | | | | DHASCO-T ® | LOVAZA ® |
|---|---|---|---|---|---|---|---|---|
| | g/kg/day fatty acid | 0.0 | 0.6 | 1.3 | 2.5 | 5.0 | 5.0 | 5.0 |
| | g/kg/day DHA (approx) | 0.0 | 0.56 | 1.2 | 2.3 | 4.7 | 2.25 | 2.09 |
| 14 | Triglycerides mg/dL | 258.5 | 168.4† | 173.6† | 120.2† | 110.7† | 123.8† | 120.6† |
| day | % of control | 100 | 65 | 67 | 47 | 43 | 48 | 47 |
| 28 | Triglycerides mg/dL | 187.1 | 174.5 | 113.1† | 112.6† | 87.6† | 125.6 | 62.9† |
| day | % of control | 100 | 93 | 60 | 60 | 47 | 67 | 35 |

^{a}Data are presented as LSMEAN.
†P < 0.05 vs control.
‡P < 0.05 vs Lovaza.

The data suggests that DHA ethyl ester (DHA-EE) reduces triglyceride levels at 14 days, even at the lowest dosage level (0.6 g/kg/day). The DHA ethyl ester reduces triglyceride levels, even in the absence of EPA. At day 28, 0.6 g DHA-EE and DHASCO-T® were not significantly lower than control. Thus, reduced amounts of DHA ethyl ester (without EPA) can be used to achieve lowered triglyceride levels similar to the dosage amounts commonly assigned to Lovaza® and DHASCO-T®.

### Example 4

### Effect of DHA Ethyl Ester on Cholesterol Levels

The effect of the DHA ethyl ester of Example 2 on cholesterol levels was investigated using male Wister rats, and was compared to DHASCO-T® and Lovaza®. The rats were administered either (a) a vehicle control, (b) DHA ethyl ester (0.6 g/kg/day, 1.3 g/kg/day, 2.5 g/kg/day, or 5.0 g/kg/day), (c) DHASCO-T (5.0 g/kg/day), or (d) Lovaza (5.0 g/kg/day) for 28 days. Cholesterol levels, i.e., total cholesterol, of the rats were measured at 14 days and 28 days. The results are presented in Table 9. Total cholesterol values shown are averages for each treatment group, i.e., actual mean values.

TABLE 9
Effect of DHA-EE, DHASCO, and Lovaza on Mean Serum Cholesterol Levels^{a}

| | | Control | DHA-EE | | | | DHASCO-T ® | LOVAZA ® |
|---|---|---|---|---|---|---|---|---|
| | g/kg/day fatty acid | 0.0 | 0.6 | 1.3 | 2.5 | 5.0 | 5.0 | 5.0 |
| | g/kg/day DHA (approx) | 0.0 | 0.56 | 1.2 | 2.3 | 4.7 | 2.25 | 2.09 |
| 14 | Cholesterol mg/dL | 91.1 ± 15.5 | 72.2 ± 17.7 | 75.0 ± 24.0 | 77.6 ± 23.1 | 67.1 ± 17.9 | 63.0 ± 20.5 | 61.4 ± 11.3 |
| day | % of control | 100 | 79 | 82 | 85 | 74 | 69 | 67 |
| 28 | Cholesterol mg/dL | 100.3 ± 47.0 | 80.3 ± 21.8 | 62.0 ± 16.0† | 71.5 ± 17.8† | 60.2 ± 19.3† | 87.2 ± 30.8 | 65.6 ± 18.4 |
| day | % of control | 100 | 80 | 62 | 72 | 60 | 87 | 66 |

^{a}Data are presented as mean ± SD
†P < 0.05 vs control
‡P < 0.05 vs Lovaza

The cholesterol results presented below in Table 10 are calculated from the same data set as in Table 9. Cholesterol level values shown in Table 10 are averages for each treatment group, presented as least squares mean values.

TABLE 10
Effect of DHA-EE, DHASCO, and Lovaza on LSMEAN Serum Cholesterol Levels^{a}

| | | Control | DHA-EE | | | | DHASCO-T ® | LOVAZA ® |
|---|---|---|---|---|---|---|---|---|
| | g/kg/day fatty acid | 0.0 | 0.6 | 1.3 | 2.5 | 5.0 | 5.0 | 5.0 |
| | g/kg/day DHA (approx) | 0.0 | 0.56 | 1.2 | 2.3 | 4.7 | 2.25 | 2.09 |
| 14 | Cholesterol mg/dL | 90.7 | 72.4 | 74.6 | 77.4 | 67.8 | 63.2 | 61.8 |
| day | % of control | 100 | 80 | 82 | 85 | 75 | 69 | 68 |
| 28 | Cholesterol mg/dL | 100.0 | 80.6 | 61.6† | 71.3† | 60.9† | 87.4 | 65.9 |
| day | % of control | 100 | 80 | 62 | 72 | 60 | 87 | 66 |

^{a}Data are presented as LSMEAN
†P < 0.05 vs control
‡P < 0.05 vs Lovaza

The data suggests that DHA-EE reduces cholesterol levels by 14 days, even at the lowest dosage level. The DHA-EE reduces cholesterol levels, even in the absence of EPA. Thus, reduced amounts of DHA (without EPA) can be used to achieve cholesterol levels significantly lower than the dosage amounts commonly assigned to Lovaza® and DHASCO-T®.

### Example 5

### Plasma Levels of DHA Ethyl Ester

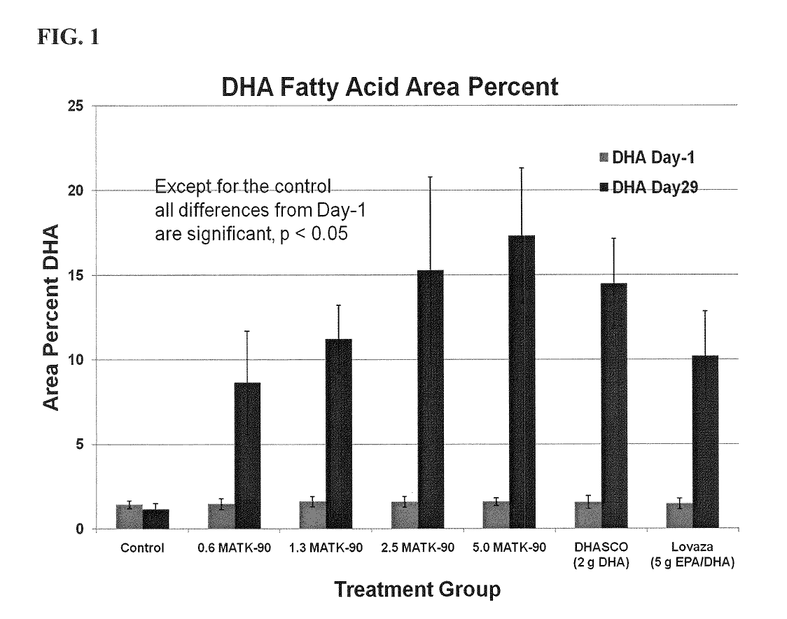

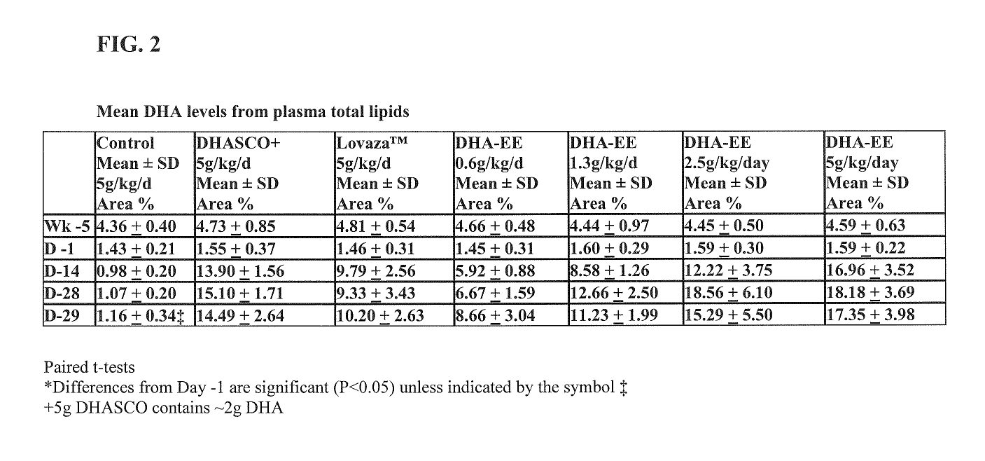

The plasma DHA fatty acid area percent and plasma EPA fatty acid area percent were determine in the rats at day 1 and day 29 (post necropsy). FIG. 1 demonstrates that plasma DHA fatty acid area percent correlates with increasing dosing of DHA, either from purified DHA-EE, DHASCO®, or Lovaza®. FIG. 2 demonstrates that plasma EPA fatty acid area percent does not increase in rats administered DHA-EE or DHASCO® to the same extent that it is increased in Lovaza®.

### Example 6

### MATK-90 900 mg Softgels Formulations Feasibility Study

The fill material (MATK-90, 900 mg/g DHA Ethyl Ester) used according to this example was a pre-blend material containing 855-945 mg/g of API (Docosahexaenoic acid or DHA Ethyl Ester) and 250 ppm of antioxidant, ascorbyl palmitate. The fill material was light sensitive and oxygen sensitive. The density of the fill material was approximately 0.9 g/ml. A mixing process was not used to formulate the DHA containing softgels prepared in this study.

### Example 7

### Feasibility Study Methods

### 1.1 Preparation of Gels

The cold crumb method was used to prepare gel mass in a small scale in the lab. The liquid excipients (water, plasticizer) and opacifiers/colorants were mixed and then stirred together with gelatin at room temperature to form a crumbly gel. The formed gel crumb was deaerated and melted at 70-80° C. in the water bath. The melted gel mass was cast on glass plates to form gel ribbons. When the ribbons cooled down, they were transferred into a drying tunnel for drying overnight. After dried, the gel swatches were ready to use.

### 1.2 Gel Compatibility (Fill/Gel-in-Vials Method)

The gel swatches were prepared by the cold crumb method. The dry gel swatches were cut into small round pieces, each piece was approximately 0.30 g in weight. One small gel piece was then placed in a glass vial which was filled with around 4.00 g of fill material. The theoretical fill to gel ratio of the softgel is 1.4 (assuming 1 g of fill weight and 0.5 g of dry gel weight). The fill material was put into the vial for this study at a higher ratio which ensured the gel piece was completely immersed in the fill. The vial was then blanketed with nitrogen and capped.

### 2 Gel Formulation Selection and Gel Compatibility Studies

### 2.1 Gel Formulation Selection

The globally acceptable excipients, bovine acid hide gelatin (195 Bloom), glycerin, and water were used in the gel formulations. The active fill material was oil based (Olgal oil). Medium and medium soft gel formulations using glycerin as plasticizer were selected. The softgel size was chosen to be 20 oblong based on the fill weight of 1000 mg (18.03 minims at density of 0.9 g/ml).

A gel compatibility study was performed using two gel formulations having the same qualitative, but slightly different quantitative, compositions in which the gel formulation referred to as “L4BX gel” contains a higher plasticizer level than the gel formulation referred to as “L3BX gel.” The detailed gel compatibility study is discussed in section 3.3 of this example.

### 2.2 Gel Color Selection

An opaque orange color capsule shell, was prepared using globally acceptable colorant/opacifies ingredients. This opaque orange color (i.e., color conversion 409P) was composed of FD&C red #40, FD&C yellow #6, and titanium dioxide. All chosen excipients were acceptable for use in orally-administered prescription products in the USA and Europe. D&C yellow #10 was initially used in the gel color formulation, but was replaced with FD&C yellow #6 based on the regulatory acceptance consideration. Titanium dioxide was used as an opacifier at a level used in other softgels intended to protect the fill from light.

### 2.3 Gel Compatibility Study

A gel compatibility study was performed using the fill/gel-in-vials method. The gels formulations can be found in Table 11.

TABLE 11
Gel and Fill Tested for Compatibility Study

| Gel/Fill | Gel | Composition of Gel |
|---|---|---|
| Tested | Formuation | Lot # of Fill |
| Gel 1 | L3BX | Gelatin (acid hide, Type 195), NF, Ph Eur |
| | | Glycerin, anhydrous 99.9%, Superol-K |
| | | USP, Ph Eur Titanium dioxide, USP/Ph Eur |
| | | FD&C Red #40 |
| | | D&C Yellow #10¹, USP Purified water |
| Gel 2 | L4BX | Gelatin (acid hide, Type 195), NF, Ph Eur |
| | | Glycerin, anhydrous 99.9%, Superol-K |
| | | USP, Ph Eur Titanium dioxide, USP/Ph Eur |
| | | FD&C Red #40 |
| | | D&C Yellow #10¹, USP |
| | | Purified water |
| Fill | DHA Purified | Lot # 5800003115EE |
| | Ethyl Ester | |

Note
¹D&C Yellow#10 was replaced with FD&C yellow #6 in the final gel formulation selection.

The fill/gel-in-vials method was used for the gel compatibility study. The compatibility samples were stored at ambient conditions for 10 days. After 10 days of storage, the gel pieces were removed from the fill, dried with lint-free Paper towel, then weighed and tested for disintegration.

The gel compatibility results are presented in Table 12. There was no significant difference in the gel weights of T0 and T10 days, which indicated that there was no migration of fill into gel or plasticizer migration into the fill material. The gel disintegration time increased slightly (+˜1 minutes), which could be analytical variation, or an indication of a slow cross linking reaction. The gel compatibility results can be found in Table 12.

TABLE 12
Gel Compatibility Results

| | T0 | | T10 days (ambient) | | |
|---|---|---|---|---|---|
| Gel | Gel weight | Disintegration | Gel weight | Disintegration | |
| Formula | (g) | time (min) | (g) | time (min) | Observation |
| L3DX | 0.29 g | N/A¹ | 0.29 g | 8 min | No sign of |
| Sample #1 | | | | | gel erosion |
| L3DX | 0.30 g | 8 min | 0.31 g | 9 min 18 sec | or brittleness |
| Sample #2 | | | | | was |
| L4DX | 0.30 g | 7 min 32 sec | 0.30 g | 9 min 18 sec | observed. |
| Sample #1 | | | | | |
| L4DX | 0.30 g | 8 min | 0.30 g | 8 min 40 sec | |
| Sample #2 | | | | | |

¹sample stuck to the disintegration vessel, no data collected

Based on this data, both gel formulations were considered compatible with the fill material. Gel formula L3BX was selected as the primary gel due to its higher gelatin content which will facilitate superior encapsulation quality. L4BX was not used as there was no evidence of brittleness.

### 2.4 Summary of Gel Formulation Selection

The gel formula L3BX with color conversion of 409P was selected as the gel formulation. A summary of L3BX can be found in Table 13.

TABLE 13
Summary of Gel Formulation with Color Conversion

| | | | |
|---|---|---|---|
| Ingredient | Use | PIN | Supplier |

L3BX@409P

| | | | |
|---|---|---|---|
| Gelatin (acid hide, | Shell Polymer | 10206-703-Y | Proprietary |
| Type 195), NF, Ph | | | information |
| Eur | | | |
| Glycerin, anhydrous | Plasticizer | 10280-001-V | Procter & Gamble |
| 99.9%, Superol-K | | | Chemicals |
| USP, Ph Eur | | | |
| Titanium dioxide, | Colorant and | 10320-001-V | Sensient Colors Inc. |
| USP/Ph Eur | opacifier | | |
| FD&C Red #40 | Colorant | 10571-001-V | Noveon |
| FD&C Yellow #6 | Colorant | 10573-001-V | Sensient Colors Inc. |
| Purified water, | Solvent | 10222-001-V | In-house |
| USP/Ph Eur | | | |

Initially the color of 015SRD (based on yellow #10) was selected, but the color was switched to color 409P (based on yellow #6) in view of the regulatory acceptance of yellow colorant. The color of 409P and 015SRD are very similar. Although the gel compatibility study was executed with color 015SRD, the gel compatibility study result is relevant to the new color considering the small amount (<3 mg per capsule) of yellow colorant used in each gel formula, and the short duration of the study.

### Example 8

### Minicap Batch Manufacturing

Two mini batches of gel capsules were produced, according to Table 14.

TABLE 14
Lot Number Summary of Minicap Batches

| Batch Lot | | |
|---|---|---|
| number | Batch Description | Fill Material |
| 1 | MATK-90 900 mg | MATK-90 (900 mg/g |
| | Softgels | DHA-EE |
| 2 | MATK-90 900 mg | Corn oil, refined |
| | Placebo Softgels | |

### 3.1 Fill Materials

The DHA-EE was manufactured by Equateq Ltd. The fill was a clear yellowish liquid, and was homogeneous. The corn oil (refined), was supplied by Henry Lamotte Oils GMBH, Lot.

### 3.2 Gel Mass Preparation

The gel was manufactured and immediately stored in the freezer prior to the minicap batch run. The gel was removed from the freezer one day prior to encapsulation, melted and heated to ˜65° C. The temperature was maintained until the encapsulation was completed.

### 3.3 Encapsulation Summary

The batches were assigned lot numbers which are provided in Table 14. Both active and placebo fills were encapsulated without further processing. Since the lab-scale encapsulation machine was equipped with an open hopper and the active fill material is sensitive to oxygen and light, a lid was placed on the hopper and both the active and placebo fill materials were encapsulated under yellow lighting with nitrogen blanketing throughout the entire encapsulation process.

The placebo fill solution (corn oil) was used to set up the machine processing parameters. Some placebo capsules were collected for analytical method development use. The theoretical in-process fill weight was 1000 mg. The potency-adjusted target in-process fill weight was 1011 mg (DHA potency 890 mg/g, as listed in the DHA Certificate of Analysis).

Table 15 summarizes the encapsulation in-process settings used during manufacturing of the MATK-90 900 mg Softgels. Fill weight and shell weight testing was performed at appropriate intervals during encapsulation. All in-process results were recorded in batch records.

TABLE 15
Minicap Machine In-Process Settings

| | | |
|---|---|---|
| Fill Material | MATK-90 (900 mg/g DHA-EE) | Corn oil, refined |
| Gel Formula | L3BX@409P | L3BX@409P |
| Dies | B20 oblong | B20 oblong |
| Wedge | W29-BS-1 | W29-BS-1 |
| Machine Speed | 2.1 rpm | 2.1 rpm |
| Pump | Standard | Standard |
| Fill Weight | Theoretical: 1000 mg | Theoretical: 1000 mg |
| | Target: 1011 mg² | Target: 1011 mg² |
| | Range: 981-1041 mg | Range: 981-1041 mg |
| | Average: 1009 mg | Average: 1012 mg |
| | % RSD: 0.34 | (n = 3) |
| | (n = 16) | |
| Shell Weight | Theoretical: 709 mg | Target: 709 mg |
| | Target: 652-765 mg | Range: 652-765 mg |
| | Average: 692 mg | Average: 694 mg |
| | % RSD: 1.57 | (n = 3) |
| | (n = 16) | |

¹Due to the different scale of the Minicap machine, these settings may not represent those required on a pilot scale encapsulation machine.
²Target fill weight was adjusted by DHA potency listed in the DHA Certificate of Analysis (890 mg/g).

### 3.4 Softgel Drying

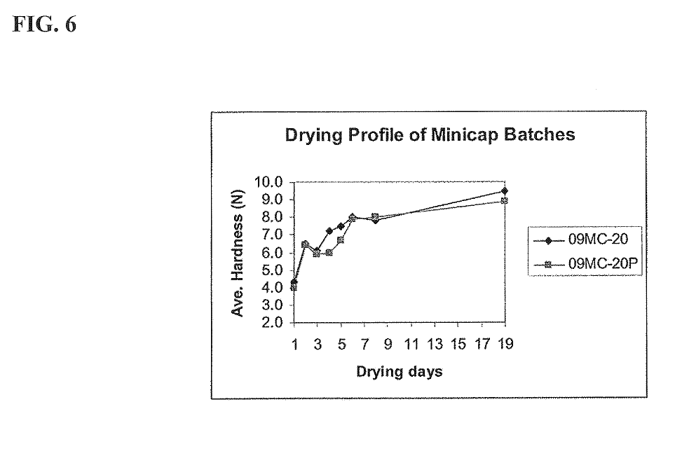

The capsules from each lot were dried in the laboratory drying tunnel. Hardness was monitored during drying process. Hardness values recorded during drying are shown in Table 16. Drying profiles are shown in FIG. 6. The drying process was stopped when the hardness of capsules reached the average hardness of 7.5-7.9N (5-6 days). A drop in hardness on day 3 could be due to the abnoimal moisture increase of drying tunnel. In order to collect information on the effect of extended drying times on the capsules physical properties, an extended drying study was started. A portion of the batch remained in the drying tunnel and the hardness was continuously monitored for up to 19 days. The results showed that the hardness increased slowly after the hardness reached 8.0N. The capsules were not brittle at the end of the extended drying study (hardness 9.5N). The hardness specification for this product was set to 7.5-9.5N. Drying profile results were also recorded in the corresponding batch records. It should be noted that the drying time for this product could change when the capsules are dried in Softgel Operations drying tunnel due to the different air flow design.

TABLE 16
Minicap Batch Hardness Results

| Drying Day | Active | Placebo |
|---|---|---|
| 1 | 4.3 | 4.0 |
| 2 | 6.5 | 6.4 |
| 3 | 6.1 | 5.9 |
| 4 | 7.2 | 6.0 |
| 5 | 7.5 | 6.7 |
| 6 | 8.0 | 7.9 |
| 8 | 7.8 | 8.0 |
| 19 | 9.5 | 8.9 |

Note
1. The number listed is the average of 5 readings.
2. Extended drying study started on day 6 for active, and day 8 for placebo.

### 3.5 Finishing (Washing/Inspecting and Packaging)

The capsules were washed by hand in a Denatured Ethanol with IPA/PHOSAL® 53 MCT (American Lecithin Company, Connecticut) wash solution to eliminate ribbon lubrication oil residue from the outside surface of the dried capsules and to apply a new lubricant to help prevent sticking. The capsules were inspected and any defects removed (and recorded in the batch record). Capsules were then allowed to dry and bulk packaged.

51 leakers, 22 defects (twin capsules) from the active batch and 21 leakers from the placebo batch were removed after washing. No other types of rejects were found during washing or bulk packaging. 368 placebo capsules were bulk packaged in poly bags. 2982 active capsules were bulk packaged in polybags and sent to Catalent Packaging Services in Philadelphia for blister packaging. 3840 active capsules were packaged into 500-cc wide mouth round HDPE bottles with white CRC and induction sealed (120 capsules per bottle). The HDPE bottles and closures were provided by Catalent Packaging Services in Philadelphia. Both packaging configurations were put on stability and tested by Catalent Analytical Services.

### Example 9

### Probe Stability

A thirty-six months informal stability study for the MATK-90 soft gelatin capsules prepared in this example was initiated for two packaging configurations (HPDE bottles and PVC blisters) studied at 25° C./60% RH and 40° C./75% RH.

### 4.1 Sample Information

1. Packaging configuration “HPDE Bottle”

MATK-90 soft gelatin capsule 900 mg strength

Container/Count: 500 cc White HDPE Bottle with CR closure, 120 count

2. Packaging configuration “PVC Blister”

MATK-90 soft gelatin capsule 900 mg strength

Container/Count: 5 (1×5) count Blister/0.010 PVC with push through lid stock)

### 4.2 Tests Performed

Various tests were performed on the HPDE Bottle configuration, and the PVC Blister configuration. The tests performed and methods used are summarized in Table 17.

TABLE 17
Stability Test Description, Method and Acceptance Criteria

| | | Acceptance |
|---|---|---|
| Test | Method | Criteria |
| Appearance | ATM-CPS-J0186 | Report Results |
| Assay/Related | ATM-MBB-J0001 (Draft | Report Results |
| Substances | | |
| for DHA | | |
| Disintegration | Current USP <701> | Report Results |
| Acid Value | EP 2.5.1 (Acid Value) | Report Results |
| Peroxide Value | EP 2.5.5 (Peroxide Value) | Report Results |
| Anisidine Value | EP 2.5.36 (Anisidine Value) | Report Results |

### 4.3 3-Month Stability Results

The Results of the information stability study of minicap batches using the HPDE

Bottle configuration and the PVC Blister configuration under various conditions is summarized in Table 18.

TABLE 18
Results of Informal Stability of Minicap Batches

| | | | | | | |
|---|---|---|---|---|---|---|
| Samples and Storage | | | | | | |
| Conditions | T₀ | T_{2 wk} | T_{4 wk} | T_{6 wk} | T_{2 m} | T_{3 m} |

Assay (Individual Result) (%)

| | | | | | | |
|---|---|---|---|---|---|---|
| Blister 25°/60% RH | 99.6, 100.2 | 98.7, 99.1 | 100.7, 99.4 | 98.1, 98.1 | 97.5, 98.1 | 98.8, 99.4 |
| Bottle 25°/60% RH | 99.6, 100.2 | Not tested | 98.9, 98.8 | Not tested | Not tested | 99.5, 97.3 |
| Blister 40° C./75% RH | 99.6, 100.2 | 99.8, 99.8 | 99.4, 100.7 | 98.7, 98.8, | 99.2, 99.5 | 101.1, 101.8 |
| | | | | 98.4, 97.8 | | |
| Bottle 40° C./75% RH | 99.6, 100.2 | Not tested | 98.7, 99.1 | Not tested | 97.0, 97.2 | 98.2, 97.5 |

Total RS for DHA (% Area

| | | | | | | |
|---|---|---|---|---|---|---|
| Blister 25° C./60% RH | 8.28 | 7.85 | 7.29 | 9.12 | 7.44 | 7.68 |
| Bottle 25° C./60% RH | 8.28 | Not tested | 8.38 | Not tested | Not tested | 7.65 |
| Blister 40° C./75% RH | 8.28 | 7.85 | 7.82 | 8.08 | 7.29 | 7.77 |
| Bottle 40° C./75% RH | 8.28 | Not tested | 7.79 | Not tested | 7.33 | 7.63 |

Disintegration Time (Rupture Time) (min)

| | | | | | | |
|---|---|---|---|---|---|---|
| Blister 25° C./60% RH | 9 | 5 | 7 | 9 | 8 | 11(7) |
| Bottle 25° C./60% RH | 9 | Not tested | 7 | Not tested | Not tested | 6(4) |
| Blister 40° C./75% RH | 9 | 5 | 10 | 10 | >25 | >45(22) |
| Bottle 40° C./75% RH | 9 | Not tested | 10 | Not tested | 13 | 30(8) |

Acid Value (mg KOH/g)

| | | | | | | |
|---|---|---|---|---|---|---|
| Blister 25° C./60% RH | 0.2 | 0.2 | 0.3 | 0.4 | 0.2 | 0.2 |
| Bottle 25° C./60% RH | 0.2 | Not tested | 0.2 | Not tested | Not tested | 0.2 |
| Blister 40° C./75% RH | 0.2 | 0.1 | 0.2 | 0.2 | 0.2 | 0.2 |
| Bottle 40° C./75% RH | 0.2 | Not tested | 0.3 | Not tested | 0.2 | 0.2 |

Peroxide Value (MegO₂/Kg)

| | | | | | | |
|---|---|---|---|---|---|---|
| Blister 25° C./60% RH | 1 | 1 | 1 | 1 | 1 | 1 |
| Bottle 25° C./60% RH | 1 | Not tested | 1 | Not tested | Not tested | 1 |
| Blister 40° C./75% RH | 1 | 1 | 1 | 1 | 1 | 1 |
| Bottle 40° C./75% RH | 1 | Not tested | 1 | Not tested | 1 | 1 |

Anisidine Value

| | | | | | | |
|---|---|---|---|---|---|---|
| Blister 25° C./60% RH | 2 | 3 | 8 | 4 | 6 | 7 |
| Bottle 25° C./60% RH | 1 | Not tested | 7 | Not tested | Not tested | 2 |
| Blister 40° C./75% RH | 2 | 2 | 5 | 7 | 21 | 22 |
| Bottle 40° C./75% RH | 2 | Not tested | 9 | Not tested | 6 | 4 |

The consistent assay/related substance, acid value and peroxide value data suggests that the product was chemically stable. However, the rise in p-Anisidine levels in the 40° C. blister sample at 3 months suggested that the oil was degrading. Disintegration results showed that the capsules increased in disintegration time as the study progressed.

The disintegration results also suggested that cross-linking of gelatin (capsule shell) occurred at the 2-month time point for the gelatin contained in the blister samples as compared to the 3-month time point for the gelatin contained in the bottled samples stored at 40° C./75% RH. The Anisidine value (AV) of blister samples significantly increased at 40° C./75% RH 2-month and 3-month time points. This increase in AV suggested that the product went through a second stage of oxidation and that non-volatile aldehydes were produced in the oil. Aldehydes are known to cause cross-linking of gelatin (capsule shell).

Thus, this comparison of the blister samples and the bottled samples, indicated that the bottled samples had better stability results (faster disintegration time, and lower Anisidine value) than the blister samples. It has been reported that softgels perform less well when stored at 40° C. relative to 25° C., since 40° C. is closer to the melting point of the shell.

### 4.4 Discussion

The fill material of the MATK-90 soft gelatin capsule 900 mg used was an oil containing 90% w/w of DHA ethyl ester. This material is oxygen sensitive and over time, may form aldehyde as a oxidation product that will react with gelatin and cause it to cross-link (forming strong bonds between the amino chains, resulting in water insoluble material that prevents capsules rupture and delays drug release from the dosage form). Exposure to high humidity and temperature are known causative factors for gelatin cross linking due to the lower permeability of gel at higher water content, and the thermodynamic effect of higher temperature.

The gel formulation selected from this study was composed of 195 bloom, acid hide gelatin. An alternative material that can be used in the gel formulations of the invention is 150 bloom gelatin which, while equally prone to the effects of cross linking, generally produces smaller increases in disintegration time. In some embodiments, the gelatin formulations used according to the invention are less prone to cross linking than gel formulations composed of 195 bloom:

In many embodiments of the invention, mixing is not required during fill of DHA-soft gelatin capsule. Accordingly, in some embodiments, the, DHA-soft gelatin capsules are directly encapsulated from an un-opened container. In other embodiments, a nitrogen blanket is applied during the encapsulation process:

The Blister packaging material used in this example was a low grade of PVC. The observed stability results indicate that this packaging material was not sufficient to protect the product from increased Anisidine values that are indicative of oxidation. Accordingly, in certain embodiments, the blister packaging material of the DHA-soft gelatin capsules of the invention have a better permeability barrier than low grade PVC. In a specific embodiment, the blister packaging material of the DHA-soft gelatin capsules of the invention is Aclar or another packaging material having a comparable or better permeability barrier than Alcar.

This study suggested that there is a range of potential viable storage conditions for the DHA-soft gelatin capsules of the invention. In particular, storage conditions of about 30° C./65% RH, about 25° C./60% RH, and about 40° C./75% RH appear to be viable storage conditions.

All of the various embodiments or options described herein can be combined in any and all variations. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above described exemplary embodiments, but should be defined only in accordance with the following claims and their equivalents.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

All publications and patent applications mentioned in this specification are herein incorporated by reference to the same extent as if each individual publication or patent application was specifically and individually indicated to be incorporated by reference in its entirety.

What is claimed is:

1. An oral dosage form comprising a gel capsule comprising:
(a) a plasticizer;
(b) a gelatin;
(c) water; and
(d) greater than 850 mg DHA ethyl ester, wherein less than about 2% (wt/wt) of the total fatty acid content of the dosage form is EPA wherein the total weight of the capsule is about 1 g.

2. The oral dosage form as recited in claim 1 wherein the plasticizer is glycerine or glycerol.

3. The oral dosage form as recited in claim 1 wherein the capsule is stable at about 25° C. at 60% relative humidity.

4. The oral dosage form as recited in claim 1 wherein the capsule is stable at about 25° C. at 60% relative humidity.

5. The oral dosage form of claim of claims 1, wherein the capsule is stable at 30° C. at 65% relative humidity.

6. The oral dosage form of claim of claims 2, wherein the capsule is stable at 30° C. at 65% relative humidity.

7. The oral dosage form of claim of claims 1, wherein the capsule is stable at 40° C. at 75% relative humidity.

8. The oral dosage form of claim of claims 2, wherein the capsule is stable at 40° C. at 75% relative humidity.
